(19) United States
(12) Patent Application Publication
(10) Pub. No.: US 2025/0213494 A1
(43) Pub. Date: Jul. 3, 2025

(54) METHODS AND COMPOSITIONS FOR POTENTIATION OF A LIGAND

(71) Applicants: Mark W. GRINSTAFF, Brookline, MA (US); Jack R. KIRSCH, Arlington, MA (US); Amanda K. WILLIAMSON, Somerville, MA (US)

(72) Inventors: Mark W. GRINSTAFF, Brookline, MA (US); Jack R. KIRSCH, Arlington, MA (US); Amanda K. WILLIAMSON, Somerville, MA (US)

(21) Appl. No.: 18/852,335

(22) PCT Filed: Mar. 27, 2023
(86) PCT No.: PCT/US2023/016465

§ 371 Date: Sep. 27, 2024

Related U.S. Application Data
(60) Provisional application No. 63/324,348, filed on Mar. 28, 2022.

(51) Int. Cl. A61K 9/50; A61K 31/167; A61K 31/573

(52) U.S. Cl. CPC A61K 9/5031; A61K 31/167; A61K 31/573

(57) ABSTRACT

Small molecule potentiators that result in the upregulation of an orthogonal receptor with the potential to enhance the efficacy of a biotherapeutic ligand to the receptor, with corticosteroids acting to potentiate RXFP1 binding ligands as an example.

The present invention claims the benefit of U.S. Provisional Application No. 63/324,348, filed Mar. 28, 2022, which is hereby incorporated by reference in its entirety and from each of which priority is claimed.

### FIELD OF THE INVENTION

The present application relates to methods and compositions for the potentiation of a ligand through modulation of its target.

### BACKGROUND OF THE INVENTION

As the scope of available therapeutics grows to treat the multitude of diseases that afflict patients, a pressing need for therapeutic potentiation has come to light. Many modern biotherapeutics rely upon specific ligand-target recognition for efficacy, which helps to minimize off-target effects and increases the therapeutic window. This benefit of specificity comes at the cost of efficacy predicated on the target of the ligand being present at a high enough density to achieve a therapeutic effect. When a patient does not respond to a given therapeutic ligand, the main option available is to increase the dose or switch to a different treatment. Something continually overlooked and undervalued in drug development is the expression and density of the ligand's target. Few mechanisms exist to address the disease-state, cell-type, or patient-specific expression of the ligand target.

As a clinically relevant, non-limiting example, this disclosure outlines the coordinated localization and potentiation of the natural antifibrotic peptide hormone, relaxin-2 (RLX) by modulation of its receptor, RXFP1, in a variety of clinically relevant tissues.

PCT Application No US2017/055799 discloses “methods for treating a stiffened joint in a subject that comprise administering relaxin, e.g., a PEGylated relaxin-2 to the subject” and “sustained release formulations in the form of a hydrogel for administering polypeptides that are covalently attached to a polymer, e.g., PEG.”

PCT Application No U.S. Ser. No. 14/380,163 discloses “Slow-release compositions . . . may be preferred to have the relaxin compounded or trapped for slow release in degradable ‘microparticles which have to be understood as solid objects of any shape, e.g. microspheres or microgranules having a median diameter of less than 250 micrometers.” and “Said slow-release composition may therefore comprise a group of microparticles made of a copolymer of the PLGA type which incorporate relaxin in the form of a water-insoluble peptide salt.”

PCT Application Publication No WO2021/237061 discloses “compositions and methods for the treatment of diseases and disorders” including wherein t″he compositions and methods involve administration of a microparticle, or composition thereof that includes an antifibrotic.”

### SUMMARY

The instant disclosure is based at least in part on the discovery that delivery of a small molecule to diseased cell type can under certain conditions result in the upregulation of an orthogonal receptor with the potential to enhance the efficacy of a different biotherapeutic ligand.

In one embodiment of any aspect herein the administration of a potentiator increases target expression, concentration, or surface density of an orthogonal target with the purpose of magnifying the biological effect of a biotherapeutic ligand at a given concentration.

In one embodiment of any aspect herein, the ability of the potentiator to increase target expression, concentration, or surface density is directly linked to a known therapeutic effect of the potentiator.

In one embodiment of any aspect herein, the ability of the potentiator to increase target expression, concentration, or surface density is indirectly linked a known therapeutic effect of the potentiator.

In one embodiment of any aspect herein, the ability of the potentiator to increase target expression, concentration, or surface density is through an unknown mechanism.

In one embodiment of any aspect herein, the administration of the potentiator decreases target expression, concentration, or surface density, with the purpose of magnifying the biological effect of the ligand at a given concentration.

In one embodiment of any aspect herein, the ability of the potentiator to decrease target expression, concentration, or surface density is directly linked to a known therapeutic effect of the potentiator.

In one embodiment of any aspect herein, the ability of the potentiator to decrease target expression, concentration, or surface density is indirectly linked a known therapeutic effect of the potentiator.

In one embodiment of any aspect herein, the ability of the potentiator to decrease target expression, concentration, or surface density is through an unknown mechanism.

In one embodiment of any aspect herein, direct therapeutic effects of the potentiator are unknown.

Another aspect details a method, wherein direct therapeutic effects of the potentiator are unknown.

In one embodiment of any of the aspects or embodiments provided herein, the potentiator is a small molecule.

In one embodiment of any of the aspects or embodiments provided herein, the potentiator is a corticosteroid. In one embodiment of any of the aspects or embodiments provided herein, the potentiator is a corticosteroid; wherein the corticosteroid is cortisol, prednisone, prednisolone, methylprednisolone, dexamethasone, betamethasone or hydrocortisone. In one embodiment of any of the aspects or embodiments provided herein, the potentiator is one or more selected from the group consisting of cortisol, prednisone, prednisolone, methylprednisolone, dexamethasone, betamethasone and hydrocortisone.

In one embodiment of any of the aspects or embodiments provided herein, the biotherapeutic ligand is an antifibrotic agent.

In one embodiment of any of the aspects or embodiments provided herein, the biotherapeutic ligand is an antifibrotic agent; wherein the antifibrotic agent is an agonist of the receptor RXFP1.

In one embodiment of any of the aspects or embodiments provided herein, the biotherapeutic ligand is an antifibrotic agent; wherein the antifibrotic agent is human relaxin-2 or an analog or variant.

In one embodiment of any of the aspects or embodiments provided herein, the biotherapeutic ligand is relaxin.

Another aspect details a method, whereby the administration of a potentiator increases the cell surface expression of RXFP1, with the purpose of magnifying the antifibrotic, vasodilatory, antifibrogenic, hemodynamic, and/or angiogenic effect of relaxin at a given concentration.

An aspect of said method, wherein the potentiator is dexamethasone; wherein the potentiator is methylprednisolone; wherein the potentiator is cortisone; wherein the potentiator is hydrocortisone; wherein the potentiator is betamethasone; wherein the potentiator is prednisolone; wherein the potentiator is prednisone; wherein the potentiator is triamcinolone; wherein the potentiator is fludrocortisone;

This disclosure lists embodiments, wherein the therapeutic effect is antifibrotic; wherein the therapeutic effect is vasodilatory; wherein the therapeutic effect is hemodynamic; wherein the therapeutic effect is angiogenic; wherein the therapeutic effect is apoptotic; wherein the therapeutic effect is antiviral; wherein the therapeutic effect increases cell proliferation; wherein the therapeutic effect is antifibrogenic; wherein the therapeutic effect is cytotoxic;

In one embodiment of any aspect herein, the potentiation and ligand treatment are commenced simultaneously

In one embodiment of any aspect herein, the potentiation treatment is commenced prior to the ligand treatment.

In one embodiment of any aspect herein, the potentiation treatment is commenced after the ligand treatment.

In one embodiment of any aspect herein, the potentiator and ligand are delivered by the same route of administration; wherein the potentiator and ligand are delivered by different routes of administration.

Other embodiments of any aspect herein detail a composition wherein the potentiator and ligand are contained within the same carrier.

In one embodiment of said aspect, the carrier is a microparticle; the carrier is a nanoparticle; the carrier is a mesh; the carrier is a polymeric buttress; the carrier is a hydrogel; the carrier is a lotion; the carrier is a cream; the carrier is a viscosupplement; the carrier is a solution;

Other embodiments of any aspect herein detail a composition wherein the potentiator and biotherapeutic ligand are contained in separate carriers.

This disclosure lists embodiments, wherein the carrier is a microparticle comprising an aliphatic polyester, biotherapeutic ligand, and potentiator, wherein (i) said microparticles have a diameter of 1-100 μm; (ii) said biotherapeutic ligand is present in an amount that is 0.01-25% of the total mass; (iii) said potentiator is present in an amount that is 0.01-25% of the total mass; (iv) said aliphatic polyester of molecular weight 10,000-200,000 Daltons.

In one embodiment of said aspect, the biotherapeutic ligand is relaxin and the potentiator is dexamethasone.

This disclosure lists embodiments, wherein the potentiator is a small molecule; wherein the potentiator is a steroid; wherein the potentiator is a nucleic acid; wherein the potentiator is a protein; wherein the potentiator is a naturally derived product; wherein the potentiator is an enzyme; wherein the potentiator is an antibody.

The following are preferred embodiments of the invention:

Embodiment 1: A method, comprising:

co-administering to a subject in need thereof a small molecule that increases expression, concentration, or cell surface density of a therapeutic target, and a ligand of the therapeutic target, wherein the therapeutic effect of the ligand is potentiated by administration of the small molecule.

Embodiment 2: A method according to embodiment 1, wherein the therapeutic target is a relaxin receptor.

Embodiment 3: A method according to embodiment 1, wherein the small molecule potentiator increases the cell surface expression of RXFP1.

Embodiment 4: A method according to one of embodiments 1-3, wherein the ligand is relaxin or a fragment thereof that binds to and induces relaxin/RXFP1 signaling.

Embodiment 5: A method according to one of embodiments 1-3, wherein the ligand is a small molecule RXFP1 agonist.

Embodiment 6: A method according to embodiment 5, wherein the ligand is 2-[[2-(1-methylethoxy)benzoyl]amino]-N-[3-[(trifluoromethyl) sulfonyl]phenyl]-benzamide or an analog thereof.

Embodiment 7: A method according to one of embodiments 1-6, wherein the small molecule potentiator is a corticosteroid.

Embodiment 8: A method according to embodiment 7, wherein the potentiator is selected from the group consisting of dexamethasone, methylprednisolone, cortisone, hydrocortisone, betamethasone, prednisolone, prednisone, triamcinolone, and fludrocortisone.

Embodiment 9: A method according to one of embodiments 1-8, wherein the small molecule and the ligand are administered simultaneously.

Embodiment 10: A method according to one of embodiments 1-8, wherein the small molecule is administered prior to administration of the ligand.

Embodiment 11: A method according to one of embodiments 1-8, wherein the small molecule is administered following administration of the ligand.

Embodiment 12: A method according to one of embodiments 1-11, wherein the small molecule and ligand are delivered by the same route of administration;

Embodiment 13: A method according to one of embodiments 1-11, wherein the potentiator and ligand are delivered by different routes of administration.

Embodiment 14: A method according to one of embodiments 1-19, wherein the small molecule and the ligand are contained within the same carrier.

Embodiment 15: A composition comprising a small molecule that increases expression, concentration, or cell surface density of a therapeutic target, and a ligand of the therapeutic target, wherein the therapeutic effect of the ligand is potentiated by administration of the small molecule

Embodiment 16: A composition according to embodiment 15, wherein the small molecule potentiator and ligand are contained within the same carrier.

Embodiment 17: A composition according to embodiment 16, wherein the carrier is selected from the group consisting of a microparticle, a nanoparticle, a mesh, a polymeric buttress, a hydrogel, a lotion, a cream, a viscosupplement, and a solution;

Embodiment 18: A composition according to embodiment 16, wherein the carrier is a microparticle comprising an aliphatic polyester, ligand, and small molecule potentiator, wherein (i) said microparticles have a diameter of 1-100 μm; (ii) said ligand is present in an amount that is 0.01-25% of the total mass; (iii) said small molecule is present in an amount that is 0.01-25% of the total mass; (iv) said aliphatic polyester has a molecular weight of between 10,000 and 200,000 Daltons.

Embodiment 19: A composition according to one of embodiments 15-18, wherein the therapeutic target is a relaxin receptor.

Embodiment 20: A composition according to embodiment 19, wherein the small molecule potentiator increases the cell surface expression of RXFP1.

Embodiment 21: A composition according to one of embodiments 15-18, wherein the ligand is relaxin or a fragment thereof that binds to and induces relaxin/RXFP1 signaling.

Embodiment 22: A composition according to one of embodiments 15-18, wherein the ligand is a small molecule RXFP1 agonist.

Embodiment 23: A composition according to embodiment 22, wherein the ligand is 2-[2-(1-methylethoxy)benzoyl]amino]-N-[3-[(trifluoromethyl) sulfonyl]phenyl]-benzamide or an analog thereof.

Embodiment 24: A composition according to one of embodiments 15-23, wherein the small molecule potentiator is a corticosteroid.

Embodiment 25: A composition according to embodiment 24, wherein the potentiator is selected from the group consisting of dexamethasone, methylprednisolone, cortisone, hydrocortisone, betamethasone, prednisolone, prednisone, triamcinolone, and fludrocortisone.

### BRIEF DESCRIPTION OF THE DRAWINGS

FIG. 1 shows disease-state specific regulation of an exemplary biotherapeutic ligand target in human fibroblast like synoviocytes. RXFP1 expression level as a function of TGF-β1 treatment. Effect of TGF-β1 (orange) treatment on RXFP1 gene expression as assessed by qPCR. * p<0.05, ** p<0.005.

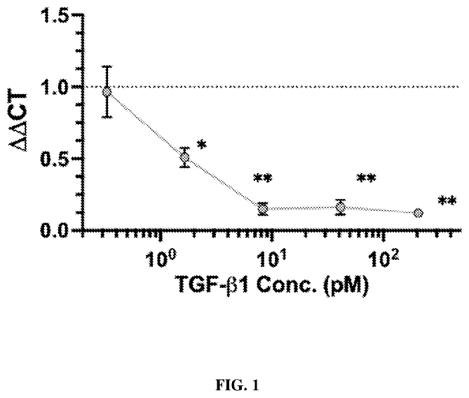

FIG. 2 shows disease-state specific regulation of an exemplary biotherapeutic ligand target in healthy and scleroderma human dermal fibroblast. (left) RXFP1 expression in isolated dermal fibroblasts from healthy patients and from patients with scleroderma, as assessed by qPCR. ΔΔCT calculation performed to cells from healthy patient N15-25. (right) RXFP1 expression as a function of TGF-β1 treatment in healthy human dermal fibroblasts. ΔΔCT calculation performed to the no treatment condition. * p<0.05, ** p<0.005, *** p<0.0005, **** p<0.0001

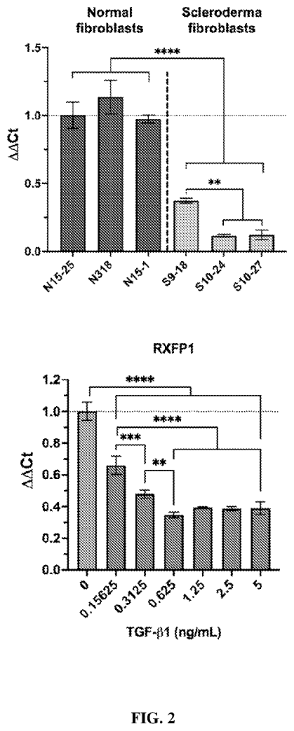

FIG. 3 shows ability of a potentiator to alter biotherapeutic ligand target gene expression in human fibroblast like synoviocytes. RXFP1 gene expression after treatment with (left) dexamethasone, (middle) methylprednisolone, and (right) cortisone under fibrotic conditions. Treatment with corticosteroid increases RXFP1 gene expression in a fibrotic state and rescues gene expression back to healthy levels. Distinct difference exists between the ability of different corticosteroids to influence RXFP1 expression.

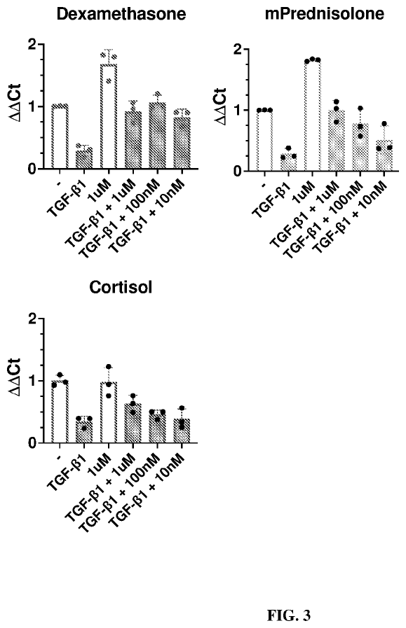

FIG. 4 shows ability of a potentiator to alter biotherapeutic ligand target gene expression in human dermal fibroblasts. (left) RXFP1 gene expression as a function of dexamethasone treatment in dermal fibroblasts isolated from a healthy human patient skin biopsy. (right) RXFP1 gene expression as a function of dexamethasone treatment in dermal fibroblasts isolated from the skin biopsy of a patient with scleroderma.

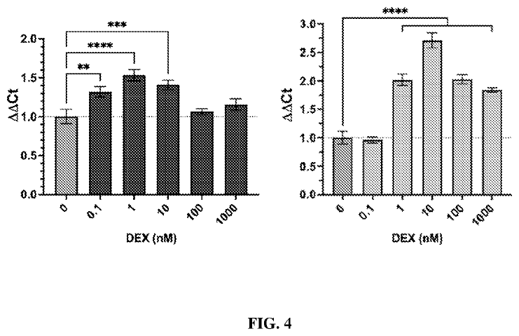

FIG. 5 shows formulations of a potentiator and of a biotherapeutic ligand in a carrier. (left) scanning electron microscopy micrographs of poly (lactide-co-glycolide) microparticles with encapsulated relaxin (RLX MPs). (right) scanning electron microscopy micrographs of poly (lactide-co-glycolide) microparticles with encapsulated dexamethasone (DEX MPs). Scale bar=10 μm.

FIG. 6 shows an in vitro RLX release study for the PLGA microparticles study performed in a biological mimetic of synovial fluid at physiological temperature with gentle agitation.

FIGS. 7A and 7B show a comparison of the internal ROM between sustained-release microspheres and instant-release injection. (A) Contracted and treated joint displays decrease in ROM from baseline after 8 weeks contracture with subsequent recovery to baseline with sIA RLX MPs and mIA RLX. (B) Healthy (contralateral) joint; no statistically significant difference for comparison of mIA RLX vs. mIA Saline or SIA RLX MPs vs. sIA Vehicle MPs. Error bars represent 95% CI; *=comparison between sIA RLX MPs and control sIA Vehicle MPs. #=comparison between mIA RLX and mIA Saline. */ #=p<0.05, **/ ##=p<0.005. Arrow color indicates corresponding to treatment arm.

FIG. 8 shows a comparison of the ROM recovery of the treated shoulder, broken down by quartile, 0-100°. (A) SIA RLX MPs (n=9), (B) SIA RLX1/3 MPs (n=4), (C) mIA RLX (n=10).

FIG. 9 shows representative H&E and safranin-O (inset) staining of coronal cross sections of humeral head after 2 weeks and 8 weeks of treatment. 10× magnification.

FIGS. 10A-10C show the structure of polymers for a RLX MP and DEX MP library. PGA, PLA, and PCL (FIG. 10A); poly (glycerol monoalkylate carbonate) s possessing C3, C8 or C18 chains (FIG. 10B); co-polymers of the poly (glycerol monoalkylate carbonate) s and PGA or PCL (FIG. 10C).

The above summary of the present invention is not intended to describe each disclosed embodiment or every implementation of the present invention. The description and examples that follows exemplifies illustrative embodiments. In several places throughout the application, guidance is provided through lists of examples, which can be used in various combinations. In each instance, the recited list serves only as a representative group and should not be interpreted as an exclusive list.

### DETAILED DESCRIPTION

The enhanced efficacy seen in top performing biotherapeutics today is due to their selective targeting of cell and tissue types. Identifying ligands for specific receptors and cellular targets offers selectivity unmatched by many traditional small molecules, and often these biotherapeutics exhibit more favorable safety profiles. However, many challenges still exist, including: 1) limited effective target-site biotherapeutic concentration; 2) pharmacokinetic barriers (e.g. short plasma half-life); and 3), reduced target receptor expression density in diseased tissue. The next-generation of drug delivery methods must account for the critical interplay between target tissue drug localization, as well as regulation of the intended receptor to maximize therapeutic efficacy.

The natural human hormone, relaxin-2 (RLX), shows promise for the treatment of musculoskeletal diseases, a class of disorders that is substantially under-represented in biotherapeutic treatments. RLX is a 6 KDa endogenous peptide hormone that acts pleiotropically with angiogenic, hemodynamic, antifibrotic and antifibrogenic activity. It is the extracellular matrix (ECM) remodeling properties of RLX that points towards its potential use as an antifibrotic therapeutic. RLX mediates the upregulation of collagen-degrading matrix metalloproteinases (MMPs) and inhibits myofibroblast differentiation through binding the ectodomain of its receptor, RXFP1. For example, RLX treatment of human fibroblasts reduces collagen types I and III and fibronectin. In a bleomycin-induced murine model of lung fibrosis, RLX decreases total pulmonary collagen content. In cultured renal fibroblasts, epithelial cells, and mesangial cells, RLX decreases TGF-β1-induced fibronectin levels and increases fibronectin degradation.

Preclinical evidence suggests that RLX is uniquely poised as a clinical antifibrotic. However, RLX failed to meet efficacy endpoints in previous phase III clinical trials that explored the use of RLX for the treatment of moderate to severe cutaneous systemic sclerosis and for the treatment of acute heart failure (AHF). In an attempt to overcome RLX's short pharmacokinetic profile (t_{1/2}=43 minutes), both the SSc and AHF trials used continuous infusion of RLX, over 24 weeks and 48 hours respectively. While continuous infusion overcomes pharmacokinetic issues, it limits the RLX concentration in target tissues due to dosing barriers associated with systemic administration. Moreover, RXFP1 expression is downregulated in many fibrotic diseases (e.g., SSc), further reducing RLX potency.

The present invention is directed in part to the use of RLX to treat shoulder arthrofibrosis, as fibrotic collagen structures are primarily responsible for the long-term range of motion (ROM) loss associated with this disease.¹⁸ Arthrofibrosis, also known as joint contracture or adhesive capsulitis, arises after trauma, surgical procedures, prolonged immobilization, or idiopathically.¹⁹⁻²¹ Approximately 15 million individuals suffer from shoulder arthrofibrosis in the U.S., with a 20% higher prevalence in women, despite no known genetic or racial bias. The predominant symptom of shoulder contracture is the gradual, painful, loss of ROM resulting from progressive fibrosis and joint capsule contraction, significantly impacting patient quality of life and productivity. Currently, recovery from shoulder contracture is arduous and protracted, with more than 1.6 million people driven to seek medical intervention each year in the U.S. Treatment options include NSAIDs, nerve blockers, or steroids to reduce pain and facilitate physical therapy (PT). These treatments may alleviate some arthrofibrosis symptoms, but they fail to address the deposition of fibrotic collagenous tissue and provide insufficient results. Advanced disease surgical interventions, such manipulation under anesthesia and capsular release, are fraught with complications and often induce further fibroses.

Advances in drug delivery such as sustained release systems and localized administration are enhancing efficacy, improving the safety margins, and in some cases revitalizing/rescuing failed therapeutics. The present application provides methods and compositions to increase target receptor density to amplify therapeutic effect. The proposed DEX and RLX co-treatment represents both a novel antifibrotic for the treatment of arthrofibrosis as well as proof of concept for increased biotherapeutic efficacy via receptor expression modulation. The innovative materials and treatment concepts are:
Establishment of the ligand binding mechanics of a unique class of G protein-coupled receptors
Demonstrating efficacy of a previously failed protein therapeutic shown to be safe and non-toxic to humans (Novartis trial for acute heart failure-see details in Aim 3)
Localized, site-specific injection of both RLX- and DEX-loaded microparticles into the synovial space via a small (≤21G) needle
Dexamethasone-based regulation of RXFP1 in fibrotic tissue
Controlled delivery of RLX and DEX via polymer engineering
Sustained co-delivery of DEX MPs and RLX MPs at a therapeutic dose over four weeks
Minimization of systemic drug exposure as a result of local delivery
Development of a dual-targeted delivery method that modulates the drug target receptor
Reduction of healthcare costs compared to surgical intervention or long-term PT/anti-inflammatory drug use
Minimally invasive procedure with treatment performed in an outpatient setting or office

Disclosed herein are methods and compositions for the potentiation of biotherapeutic ligand targets for magnifying and potentiating the efficacy of administered biotherapeutic ligands. The technology disclosed herein allows for overcoming previous clinical failures due to lack of efficacy by increasing the relative impact of a given dose. Advantageously, it also allows for use of a biotherapeutic ligand in a disease state where the biotherapeutic ligand target is at concentrations too low to allow for efficacy. Additionally, and more generally, the technology disclosed herein increases the efficacy of a biotherapeutic ligand without necessitating an increased dose. This treatment strategy is applicable to the treatment of various diseases, injuries, traumas, infections and health conditions. Non limiting examples include cancer, stroke, diabetes mellitus, cirrhosis, pneumonia, respiratory infections and autoimmune, ischemic heart, coronary artery, chronic obstructive pulmonary, and musculoskeletal diseases.

### Antifibrotic Agents

Described herein include methods and compositions that relate to use of an antifibrotic agent.

In one embodiment, the antifibrotic agent is an agonist of the receptor RXFP1. In one embodiment, the antifibrotic agent is human relaxin-2 or an analog or variant.

The term “relaxin” as used herein, refers to a polypeptide belonging to the relaxin family (e.g., relaxin-2), a relaxin analog (e.g., a polypeptide that binds to a relaxin receptor), or a fragment (e.g., a bioactive fragment) or variant of any of the foregoing and/or any agent that is an agonist of an agent that binds the relaxin receptor family of proteins (RXFP1, RXFP2, RXFP3, RXFP4).

Relaxin is an approximately 6-kDa protein belonging to the insulin superfamily (Sherwood O.D., Endocr. Rev. 2004, 25 (2): 205-34). Like insulin, relaxin is processed from a prepro-form to the mature hormone, containing A and B peptide chains connected by two interchain disulfide bridges and one intrachain disulfide within the A chain (Chan L. J. et al., Protein Pept. Lett. 2011, 18 (3): 220-9). Relaxin readily decreases collagen secretion and increases collagen degradation by increasing the expression of MMPs and decreasing the expression of TIMPs (Samuel C. S. et al., Cell Mol. Life Sci. 2007, 64 (12): 1539-57). This hormone is involved in reproduction, where it inhibits uterine contraction and induces growth and softening of the cervix to assist child delivery (Parry L. J. et al., Adv. Exp. Med. Biol. 2007, 612:34-48). Recently, a highly purified recombinant form of H2 relaxin, or human relaxin-2, has been tested in a number of in vitro and in vivo systems to evaluate both its ability to modify connective tissue and its potential antifibrotic properties. Several studies report that relaxin-2 acts at multiple levels to inhibit fibrogenesis and collagen overexpression associated with fibrosis and is able to prevent and treat pulmonary, renal, cardiac, and hepatic fibrosis (Bennett R. G., Transl. Res. 2009, 154 (1): 1-6). Relaxin treatment of human fibroblasts caused a reduction in levels of collagen types I and III and fibronectin (Unemori E. N. et al., The Journal of Clinical Investigation 1996, 98 (12): 2739-45). In vivo, relaxin-2 decreased collagen build-up in the lung induced by bleomycin and improved the overall amount of fibrosis (Unemori E. N. et al., The Journal of Clinical Investigation 1996, 98 (12): 2739-45). In cultured renal fibroblasts, epithelial cells and mesangial cells, relaxin-2 decreased TGF-β-induced fibronectin levels and increased fibronectin degradation (McDonald G. A. et al., American Journal of Physiology Renal Physiology 2003, 285 (1): F59-67). Relaxin-2 has been shown to have a rapid pharmacokinetic profile with a plasma half-life of minutes. Previous clinical trials investigating relaxin-2 as a treatment for scleroderma, acute heart failure, and for the induction of labor through cervical ripening, utilized continuous infusion of relaxin-2 via either intravenous adminstration or subcutaneous administration through a minipump. These clinical studies failed to meet their clinical endpoints because of insufficient relaxin receptor activation even though the dose was in the mg per kg range. Efficacy of relaxin-2 requires activation of a transmembrane relaxin receptor for downstream signalling. Other clinical trials utilized continuous infusion in an attempt to overcome pharmacokinetic limitations but also failed. The localized, sustained release of relaxin-2 achieves sustained receptor activation without necessitating continuous administration.

Unless specified to the contrary, the term “relaxin” as used herein encompasses a relaxin or an analog, a fragment (e.g., a bioactive fragment) or a variant thereof. The term “relaxin or an analog, a fragment or a variant thereof” encompasses any member of the relaxin-like peptide family which belongs to the insulin superfamily. The relaxin-like peptide family includes relaxin-like (RLN) peptides, e.g., relaxin-1 (RLN1), relaxin-2 (RLN2) and relaxin-3 (RLN3), and the insulin-like (INSL) peptides, e.g., INSL3, INSL4, INSL5 and INSL6. Representative sequences of human RLN1 are listed herein as SEQ ID NOS: 4-7; representative sequences of human RLN2 are listed herein as SEQ ID NOS: 1-3; representative sequences of human RLN3 are listed herein as SEQ ID NOS: 8-10; a representative sequence of human INSL3 is listed herein as SEQ ID NO: 11; representative sequences of human INSL4 are listed herein as SEQ ID NOS: 12-13; representative sequences of human INSL5 are listed herein as SEQ ID NOS. 14-15; and a representative sequence of human INSL6 is listed herein as SEQ ID NO: 16. In some embodiments, the term “relaxin or an analog, a fragment or a variant thereof may encompass any polypeptide having at least 70%, e.g., at least 75%, at least 80%, at least 85%, at least 90%, at least 95%, 96%, 97%, 98% or at least 99% sequence identity with any of SEQ ID NOS: 1-16, as well as any polypeptide sequence that comprises any of SEQ ID NOS: 1-16. In one embodiment of the disclosure, the relaxin includes RLN1, RLN2 or RLN3. In one embodiment, the relaxin is relaxin-1. In another embodiment, the relaxin is relaxin-3. In a preferred embodiment, the relaxin is relaxin-2. In another embodiment of the disclosure, the relaxin includes INSL3, INSL4, INSL5 or INSL6. In one embodiment, the relaxin is INSL3. In one embodiment, the relaxin is INSL4. In one embodiment, the relaxin is INSL5. In one embodiment, the relaxin is INSL6.

In some embodiments, the relaxin is recombinantly produced, for example in a bacterial, mammalian or yeast host cell. In other aspects the relaxin has been fully or partially chemically synthesized.

In some embodiments, the term relaxin encompasses any natural, synthetic, or semi-synthetic composition that is capable of interacting with a relaxin family protein receptors (RXFP1, RXFP2, RXFP3, RXPF4) that impacts the form, function, or activity of the receptor. These compounds include but are not limited to native relaxin-2, relaxin-2 variants, polypeptides, DNA or RNA polynucleotides, small molecules, as well as any of the previously listed compounds conjugated to, or associated with, the relaxin-2 protein.

The term “relaxin or an analog, a fragment or a variant thereof” may also encompass any member the relaxin-like peptide family includes relaxin-like (RLN) peptides, e.g., relaxin-1 (RLN1), relaxin-2 (RLN2) and relaxin-3 (RLN3), and the insulin-like (INSL) peptides, e.g., INSL3, INSL4, INSL5 and INSL6. Representative sequences of human RLN1 are listed herein as SEQ ID NOS: 4-7; representative sequences of human RLN2 are listed herein as SEQ ID NOS: 1-3; representative sequences of human RLN3 are listed herein as SEQ ID NOS: 8-10; representative sequence of human INSL3 is listed herein as SEQ ID NO: 11; representative sequences of human INSL4 are listed herein as SEQ ID NOS: 12-13; representative sequences of human INSL5 are listed herein as SEQ ID NOS. 14-15; and representative sequence of human INSL6 is listed herein as SEQ ID NO: 16. The term “relaxin or an analog, a fragment or a variant thereof” also in some embodiments encompasses any polypeptide having at least 70%, e.g., at least 75%, at least 80%, at least 85%, at least 90%, at least 95% or at least 99% sequence identity with any of SEQ ID NOS: 1-16, as well as any polypeptide sequence that comprises any of SEQ ID NOS: 1-16. In one embodiment of the formulation, the relaxin includes RLN1, RLN2 or RLN3. In one embodiment, the relaxin is relaxin-2. In another embodiment, the relaxin includes INSL3, INSL4, NSL5 or INSL6.

The term “relaxin or an analog, a fragment or a variant thereof” also in some embodiments may encompass any mutant member of the relaxin-like peptide family. Such mutant may be, e.g., a RLN1, RLN2, RLN3, INSL3, INSL4, INSL5 or INSL6 comprising one or more mutations, e.g., substitutions, additions or deletions of one or more amino acids (native or non-native) in the known sequence of RLN1, RLN2, RLN3, INSL3, INSL4, INSL5 or INSL6. For example, a mutant member of the relaxin-like peptide family may comprise any naturally occurring or artificially produced variants of RLN1, RLN2, RLN3, INSL3, INSL4, INSL5 or INSL6.

The term “relaxin fragment” or “a fragment of relaxin” as used herein encompasses a fragment of a relaxin, i.e., a partial sequence of any member of the relaxin-like peptide family, that retains its ability to treat stiffened joints through interaction with the relaxin family receptors. Examples include those sequences described in European Patent Office Application No. EP1641824B1 (Relaxin superfamily peptide analogues), the entire contents of which are incorporated herein by reference.

The term “relaxin analog” or an “analog of relaxin” includes any non-relaxin polypeptide sequence that possesses the biological activity of relaxin, i.e., the ability to interact with the relaxin family receptors. In one embodiment, such polypeptide sequence may comprise prolactin or an analog, a fragment or a variant thereof. In another embodiment, such sequence may comprise the truncated B-chain analogue of relaxin known as B7-33, described in ACS Appl. Mater. Interfaces 2019, 11, 49, 45511-45519.

In some embodiments, the term agent or “relaxin analog” also includes a relaxin receptor agonist, e.g., any agent, such as a small molecule, a polypeptide, a polynucleotide or a polysaccharide, that can bind to and activate a relaxin receptor, e.g., one or more of RXFP1, RXFP2, RXFP3 and RXFP4. For example, a relaxin receptor agonist may be a polypeptide comprising the receptor binding site of relaxin. A relaxin receptor agonist may also be a polypeptide comprising any other sequence capable of binding to and activating the relaxin receptor, e.g., RXFP1, RXFP2, RXFP3 and RXFP4. Other examples include those agonists described in US Patent Application No. US20130237481A1 (Modified relaxin polypeptides and their uses), US Patent Application No. US20180222960A1 (Modified relaxin polypeptides comprising a pharmacokinetic enhancer and uses thereof), US Patent Application No. U.S. Pat. No. 8,445,635B2 (Modified H2 relaxin for tumor suppression), European Patent Office Application No. EP3067365A1 (Human relaxin analogue, pharmaceutical composition of same, and pharmaceutical application of same), and US Patent Application No. US20180222960A1 (Modified relaxin polypeptides comprising a pharmacokinetic enhancer and uses thereof) the entire contents of which are incorporated herein by reference.

The term “relaxin or an analog, a fragment or a variant thereof” includes any recombinantly produced relaxin, such as, e.g., Serelaxin (RLX030) developed by Novartis. Methods for producing recombinant relaxin, e.g., relaxin-2, are described, .e.g., in U.S. Pat. No. 5,464,756, the entire contents of which are incorporated herein by reference. The recombinantly produced relaxin or analog, fragment or variant thereof may comprise a relaxin sequence, e.g., RLN1, RLN2, RLN3, INSL3, INSL4, INSL5 or INSL6, and a histidine (His) tag to aid in the purification of the relaxin after being recombinantly produced.

The relaxin or analog, fragment or variant thereof may also comprise one or more chemical modifications, e.g., chemical groups covalently attached to the relaxin or an analog, a fragment or a variant thereof. Such chemical groups may include, e.g., carbohydrates or other polymers, e.g., polyethylene glycol (PEG), e.g., polypeptide, e.g. one or more lipids (Design and Synthesis of Potent, Long-Acting Lipidated Relaxin-2 Analogs, Bioconjugate Chem. 2019, 30, 1, 83-89). Other examples include fragments or variants described in US Patent Application No. US2018/0326079 (NOVELFATTYACIDS AND THEIR USE IN CONJUGATION TO BIOMOLECULES), U.S. Pat. No. 9,931,372B2 (SYNTHETIC APELIN FATTYACID CONJUGATES WITH IMPROVED HALF-LIFE), the entire contents of which are incorporated herein by reference.

In some embodiments, relaxin or an analog, a fragment or a variant thereof is co-administered with ML290 or its analog, fragment or a variant to prolong or enhance the effects of RXLP1 activation (Kocan, M., et al. Sci. Rep., 2017).

In some embodiments, the term relaxin includes relaxin attached, e.g., covalently attached, to an immunoglobulin or a fragment of an immunoglobulin, e.g., an antibody or a fragment of an antibody, for example, the immunoglobulin fusion proteins described in WO 2017/100540. In other embodiments, the term relaxin does not include relaxin attached, e.g., covalently attached, to an immunoglobulin or a fragment of an immunoglobulin.

The components of the combination therapy of the present invention are administered in a therapeutically effective amount. As used herein, the term “therapeutically effective amount” or “effective amount” refers to the combination therapy that, when administered to a cell, tissue, or subject is effective to prevent or ameliorate the disease or condition to be treated or one or more of its symptoms. Thus, a therapeutically effective dose can refer to that amount of the active agent(s) sufficient to result in amelioration of symptoms, e.g., treatment, healing, prevention or amelioration of the relevant medical condition, or an increase in rate of treatment, healing, prevention or amelioration of such conditions. When applied to an individual active agent administered alone, a therapeutically effective dose refers to that ingredient alone. When applied to a combination, a therapeutically effective dose refers to combined amounts of the active agents that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

When active agents are administered in combination, separate dosage forms of the active agents can be administered to the subject or a single dosage form comprising both active agents can be administered to the subject. If administered as a separate dosage form, the therapeutic agents may be administered simultaneously or sequentially (in either order). Administration of two or more agents in combination can also be referred to herein as “co-administration.” Methods for co-administration or treatment with a second therapeutic agent are well known in the art, see, e.g., Hardman, et al. (eds.), 2001, Goodman and Gilman's The Pharmacological Basis of Therapeutics, 10th ed., McGraw-Hill, New York, NY; Poole and Peterson (eds.), 2001, Pharmacotherapeutics for Advanced Practice: A Practical Approach, Lippincott, Williams & Wilkins, Phila., PA; Chabner and Longo (eds.), 2001, Cancer Chemotherapy and Biotherapy, Lippincott, Williams & Wilkins, Phila., PA.

### Microparticles, and Compositions and Formulations Thereof

Aspects described herein relate to a microparticle comprising an aliphatic polyester and an antifibriotic agent. In some embodiments the disclosures described herein relate to a microparticle comprising an aliphatic polyester and a potentiator as described herein. In some embodiments the disclosures described herein relate to a microparticle comprising an aliphatic polyester and a antifibrotic agent and potentiator as described herein. Exemplary aliphatic polyesters include poly-lactide-co-glycolide, or polycaprolactone.

In one embodiment, the microparticle further comprises a vinyl polymer. Exemplary vinyl polymers include poly (vinyl alcohol) or poly (pyrrolidone).

Another aspect herein is a microparticle comprising an aliphatic polyester and an antifibrotic agent, the microparticles have a diameter of 1-100 μm.

Another aspect herein is a microparticle comprising an aliphatic polyester and an antifibrotic agent, the antifibrotic agent is relaxin and is present in an amount that is 0.01-10% of total mass.

Another aspect herein is a microparticle comprising an aliphatic polyester and an antifibrotic agent, the aliphatic polyester is of molecular weight 10,000-200,000 daltons.

Another aspect herein is a microparticle comprising an aliphatic polyester, a vinyl polymer and an antifibrotic agent.

Another aspect herein is a microparticle comprising an aliphatic polyester, a vinyl polymer and an antifibrotic agent, the microparticles have a diameter of 1-100 μm.

Another aspect herein is a microparticle comprising an aliphatic polyester, a vinyl polymer and an antifibrotic agent, the antifibrotic agent is relaxin and is present in an amount that is 0.01-10% of total mass.

Another aspect herein is a microparticle comprising an aliphatic polyester, a vinyl polymer and an antifibrotic agent, the aliphatic polyester is of molecular weight 10,000-200,000 daltons.

Another aspect herein is a PLGA microparticle comprising an aliphatic polyester and an antifibrotic agent, the microparticles have a diameter of 1-100 μm.

Another aspect herein is a PLGA microparticle comprising an aliphatic polyester and an antifibrotic agent, the antifibrotic agent is relaxin and is present in an amount that is 0.01-10% of total mass.

Another aspect herein is a PLGA microparticle comprising an aliphatic polyester and an antifibrotic agent, the aliphatic polyester is of molecular weight 10,000-200,000 daltons.

Another aspect herein is a PLGA microparticle comprising an aliphatic polyester, a vinyl polymer and an antifibrotic agent, the microparticles have a diameter of 1-50 μm.

Another aspect herein is a PLGA microparticle comprising an aliphatic polyester, a vinyl polymer and an antifibrotic agent, the antifibrotic agent is relaxin and is present in an amount that is 0.1-10% of total mass.

Another aspect herein is a PLGA microparticle comprising an aliphatic polyester, a vinyl polymer and an antifibrotic agent, the aliphatic polyester is of molecular weight 10,000-200,000 daltons.

### Pharmaceutical Compositions

Various aspects herein relate to a composition comprising any of the microparticles described herein. In various embodiments, the composition is a pharmaceutical composition.

As used herein, the term “pharmaceutical composition” can include any material or substance that, when combined with an active ingredient (e.g., an antifibrotic agent, such as relaxin), allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. The term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

In some embodiments, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs. In addition, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the agents described herein are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The agent can also be in micro-encapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms, the agent can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

The present disclosure provides sustained release formulations for delivering a polypeptide therapeutic to a subject in need thereof. The sustained release formulations of the disclosure consist of a hydrogel, microparticle or some matrix encapsulation of the agent. One example of the agent is relaxin. The sustained release comprises the agent e.g., relaxin encapsulated by or chemically bound to the depot support material via a linker. The linker may, comprise a polymer, a non-cleavable linker, or a cleavable linker, either through chemical or enzymatic means. The depot may be formed in situ following mixing of the agent with the material. The depot may be formed prior to mixing of the relaxin with the material.

The sustained release formulation comprising the agent e.g., relaxin may be in the form of a hydrogel or microparticle which comprises one or more polymers. The polymers that may be used in a sustained release relaxin formulation may include, without limitation, polyethylene glycol (PEG), alginate, agarose, poly (ethylene glycol dimethacrylate), polylactic acid, polyglycolic acid, poly-lactide-co-glycolide, gelatin, collagen, agarose, pectin, poly (lysine), polyhydroxybutyrate, poly-epsilon-caprolactone, polyphosphazines, poly (vinyl alcohol), poly (alkylene oxide), poly(ethylene oxide), poly (allylamine), poly (acrylate), poly (4-aminomethylstyrene), pluronic polyol, polyoxamer, poly (uronic acid), poly (anhydride), poly(vinylpyrrolidone), bolaamphiphiles, glycosyl-nucleosides, and fluorocarbon chains.

In some embodiments of any of the aspects described herein, an agent is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

In some embodiments, any aforementioned polymers, prior to or after loading of relaxin, may be characterized (e.g. size, molecular weight, charge, secondary structure, and purity) by techniques including, but not limited to, gel permeation chromatography, high performance liquid chromatography, ultra-performance liquid chromatography, MALDI-TOF mass spectroscopy, viscometry, and light scattering (e.g. multi-angle, low angle laser).

In some embodiments, the rate of release of relaxin may be characterized by techniques including, but not limited to, high performance liquid chromatography, ultra-performance liquid chromatography, fast protein liquid chromatography, enzyme linked immunosorbent assay, and ligand binding assay. In some embodiments, the release rate of relaxin is measured as the concentration of relaxin in any biologically relevant liquid solution or suspension or medium (e.g. saline, mammalian cell culture media, synthetic synovial fluid, synovial fluid, serum, synthetic serum, plasma, synthetic plasma and deionized water) that the formulation is also in. In specific embodiments, the formulation and biologically relevant liquid solution or suspension is maintained at a specific temperature. In specific embodiments, the formulation and biologically relevant liquid solution or suspension is agitated or mixed at a set or varying rate of motion. In specific embodiments, the concentration of relaxin released into the biologically relevant liquid solution or suspension is measured using a direct enzyme linked immunosorbent assay. In specific embodiments, the concentration of relaxin released into the biologically relevant liquid solution or suspension is measured using an indirect enzyme linked immunosorbent assay. In specific embodiments, the concentration of relaxin released into the biologically relevant liquid solution or suspension is measured using a sandwich enzyme linked immunosorbent assay. In a preferred embodiment, the concentration of relaxin released into the biologically relevant liquid solution or suspension is measured using the Human Relaxin-2 Quantikine ELISA Kit from Bio-techne corporation.

In some embodiments, the size and morphology (e.g. diameter, sphericity, and porosity) of relaxin microparticles may be characterized by techniques including, but not limited to, dynamic light scattering, coulter counter, microscopy, sieve analysis, dynamic image analysis, static image analysis, and laser diffraction.

In some embodiments, the total loaded content of relaxin in relaxin microparticles (e.g. percent of relaxin as weight/volume, percent of relaxin as weight/weight) may be characterized by techniques including, but not limited to, mass balance, limited to, high performance liquid chromatography, ultra-performance liquid chromatography, fast protein liquid chromatography, enzyme linked immunosorbent assay, and ligand binding assay. In some embodiments, the formulation may be purified and dissolved to assess total loaded content of relaxin.

In some embodiments, the total loaded content (i.e. mass) of relaxin in relaxin microparticles is measured as the concentration of relaxin in any liquid solution, suspension or medium (e.g. saline, mammalian cell culture media, synthetic synovial fluid, synovial fluid, serum, synthetic serum, plasma, synthetic plasma, methylene chloride, acetonitrile, ethyl acetate, and deionized water) that the total formulation may be dissolved in. In specific embodiments, the concentration of relaxin after formulation dissolution in the liquid solution, suspension, or medium is measured using a direct enzyme linked immunosorbent assay. In specific embodiments, the concentration of relaxin after formulation dissolution in the liquid solution, suspension, of medium is measured using an indirect enzyme linked immunosorbent assay. In specific embodiments, the concentration of relaxin after formulation dissolution in the liquid solution, suspension, of medium is measured using a sandwich enzyme linked immunosorbent assay. In a preferred embodiment, the concentration of relaxin after formulation dissolution in the liquid solution, suspension, of medium is measured using the Human Relaxin-2 Quantikine ELISA Kit from Bio-techne corporation.

In certain embodiments, the sustained release formulation comprises of PEG, e.g., a linear PEG or a branched PEG. In certain embodiments, the molecular weight of the PEG is more than 0.2 kDa, more than 0.5 kDa, more than 1 kDa, more than 5 kDa, more than 10 kDa, or more than 20 kDa

In some embodiments, the hydrogel comprises of PEG-based crosslinkers with an internal thioester that will be reacted with dendrons to prepare hydrogels. These hydrogels may be prepared in varying weight percent to modulate mechanical properties. In specific embodiments the internal thioester allows for controlled dissolution through the use of a cysteine methyl ester solution. In specific embodiments, the gels material properties including, but not limited to, release profile, young's modulus, sheer modulus, hydrophobicity, and, elasticity can be varied through modification of the thioester moiety to modulate material properties of hydrogel.

In one embodiment of any aspect herein, the aliphatic polyester is poly-lactide-co-glycolide.

In one embodiment of any aspect herein, the aliphatic polyester is polycaprolactone.

In one embodiment of any aspect herein, the aliphatic polyester is of molecular weight 10,000-200,000 daltons; 10,000-150,000 daltons; or 25,000-125,000 daltons; or 40,00-100,000 daltons; 10,000-30,000 daltons; 30,000-50,000 daltons; 50,000-70,000 daltons; 70,000-90,000 daltons; 90,000-120,000 daltons; or 120,000-150,000 daltons.

In one embodiment of any aspect herein, the aliphatic polyester is terminated by an ester functional group.

In one embodiment of any aspect herein, the aliphatic polyester is terminated by an alkyl-ester functional group.

In one embodiment of any aspect herein, the aliphatic polyester is terminated by a carboxylic acid functional group.

In one embodiment of any aspect herein, the aliphatic polyester is terminated by an amine functional group. In one embodiment of any aspect herein, the formulation comprises a vinyl polymer that is poly (vinyl alcohol).

In one embodiment of any aspect herein, the formulation comprises a vinyl polymer that is poly (pyrrolidone).

In one embodiment of any aspect herein, the formulation comprises a vinyl polymer that is of molecular weight 10,000-200,000 daltons; 10,000-150,000 daltons; or 25,000-125,000 daltons; or 40,00-100,000 daltons; 10,000-30,000 daltons; 30,000-50,000 daltons; 50,000-70,000 daltons; 70,000-90,000 daltons; 90,000-120,000 daltons; or 120,000-150,000 daltons.

In one embodiment of any aspect herein, the diameter of the microparticles is 1-100 μm.

In one embodiment of any aspect herein, the diameter of the microparticles is 1-75 μm; or 1-50 μm; or 5-50 μm; or 25-50 μm; or 30-50 μm; or 40-50 μm; or 5-10 μm; 5-8 μm; 8-12 μm; 12-18 μm; 18-25 μm; 25-35 μm; 35-45 μm; 45-50 μm; 1 μm; 2 μm; 3 μm; 4 μm; 5 μm; 6 μm; 7 μm; 8 μm; 9 μm; 10 μm; 15 μm; 20 μm; 25 μm; 30 μm; 35 μm; 40 μm; 45 μm; 50 μm; 75 μm; 100 μm; 150 μm; or 200 μm.

In one embodiment of any aspect herein, the aliphatic polyester is poly-lactide-co-glycolide with a molar ratio of 15:85-25:75, lactide:glycolide; poly-lactide-co-glycolide with a molar ratio of 25:75-35:65, lactide:glycolide; poly-lactide-co-glycolide with a molar ratio of 35:65-45:55, lactide:glycolide; poly-lactide-co-glycolide with a molar ratio of 45:55-55:45, lactide:glycolide; poly-lactide-co-glycolide with a molar ratio of 55:45-65:35, lactide:glycolide; poly-lactide-co-glycolide with a molar ratio of 65:35-75:25, lactide:glycolide; poly-lactide-co-glycolide with a molar ratio of 75:25-85:15, lactide:glycolide; poly-lactide-co-glycolide with a molar ratio of about 50:50, lactide:glycolide; poly-lactide-co-glycolide with a molar ratio of about 45:55, lactide:glycolide; poly-lactide-co-glycolide with a molar ratio of about 55:45, lactide:glycolide; poly-lactide-co-glycolide with a molar ratio of about 40:60, lactide:glycolide; or poly-lactide-co-glycolide with a molar ratio of about 60:40, lactide:glycolide.

In one embodiment of any aspect herein, the formulation comprises a vinyl polymer that is about 0.01-0.1% of total mass; 0.1-0.3% of total mass; 0.2-0.9% of total mass; 0.3-0.7% of total mass; 0.4-0.6% of total mass; 0.3-0.6% of total mass; 0.6-1.0% of total mass; 1.0-5.0% of total mass; 5.0-10.0% of total mass; 10.0-30.0% of total mass; 0.1% of total mass; 0.2% of total mass; 0.3% of total mass; 0.4% of total mass; 0.5% of total mass; 0.6% of total mass; 0.7% of total mass; 0.8% of total mass; 0.9% of total mass; 10% of total mass; 15% of total mass; 20% of total mass; 25% of total mass; 30% of total mass.

In one embodiment of any aspect herein, the antifibrotic agent is 0.005-5% of the total formulation mass. In one embodiment of any aspect herein, the antifibrotic agent is 0.01-10%; or 0.1-5% of the total formulation mass; or 0.2-4% of the total formulation mass; or 0.3-3% of the total formulation mass; or 0.5-2% of the total formulation mass; or 0.5-1.5% of the total formulation mass; or 0.5-3% of the total formulation mass; or 1-2% of the total formulation mass; or 1-5% of the total formulation mass; or 3-7% of the total formulation mass; or 5-10% of the total formulation mass.

In one embodiment of any aspect herein, the antifibrotic agent is about 0.005-0.01% of the total formulation mass; 0.01-0.05% of the total formulation mass; 0.05-0.1% of the total formulation mass; 0.1-0.5% of the total formulation mass; 0.5-1.0% of the total formulation mass; 1.0-2.5% of the total formulation mass; 2.5-5.0% of the total formulation mass; 0.25% of the total formulation mass; 0.5% of the total formulation mass; 0.75% of the total formulation mass; 1% of the total formulation mass; 1.25% of the total formulation mass; 1.5% of the total formulation mass; 1.75% of the total formulation mass; 2% of the total formulation mass; 2.5% of the total formulation mass; 3% of the total formulation mass; or 5% of the total formulation mass.

In one embodiment of any aspect herein, the formulation comprises PLGA microparticles with a PLGA molar ratio that is about 50:50 lactide:glycolide, a relaxin loaded at about 1% by weight of the microparticles, and PVA in a concentration of about 0.5% by weight.

In one embodiment of any aspect herein, the formulation comprises PLGA microparticles with a PLGA molar ratio that is about 50:50 lactide:glycolide, a relaxin loaded at about 1% by weight of the microparticles and PVA in a concentration of about 0.0% by weight

In one embodiment of any aspect herein, the formulation comprises PLGA microparticles with a PLGA molar ratio that is about 60:40 lactide:glycolide, a relaxin loaded at about 1% by weight of the microparticles, and PVA in a concentration of about 0.5% by weight.

In one embodiment of any aspect herein, the formulation comprises PLGA microparticles with a PLGA molar ratio that is 40:60 lactide:glycolide, a relaxin loaded at about 1% by weight of the microparticles, and PVA in a concentration of about 0.5% by weight.

In one embodiment of any aspect herein; the formulation comprises microparticles suspended in a liquid solution.

In one embodiment of any aspect herein; the formulation comprises microparticles suspended in a sodium chloride liquid solution.

In one embodiment of any aspect herein; the formulation comprises microparticles suspended in a sodium chloride liquid solution; the sodium chloride is 0.5-1.5 w/w %; or between 0.75-1.25 w/w %; or about 0.5 w/w %; or about 0.6 w/w %; or about 0.7 w/w %; or about 0.8 w/w %; or about 0.9 w/w %; or about 1.0 w/w %; or about 1.1 w/w %; or about 1.2 w/w %; or about 1.3 w/w %; or about 1.4 w/w %; or about 1.5 w/w % of the liquid solution.

In one embodiment of any aspect herein; the formulation comprises microparticles suspended in a sodium carboxymethylcellulose solution.

In one embodiment of any aspect herein, the formulation comprises microparticles suspended in a sodium carboxymethylcellulose solution; the sodium carboxymethylcellulose solution is 0.1-1.0 w/w %; or between 0.25-0.75 w/w %; or about 0.1 w/w %; or about 0.2 w/w %; or about 0.3 w/w %; or about 0.4 w/w %; or about 0.5 w/w %; or about 0.6 w/w %; or about 0.7 w/w %; or about 0.8 w/w %; or about 0.9 w/w %; or about 1.0 w/w % of the liquid solution.

In one embodiment of any aspect herein, the formulation is a sustained release formulation.

In one embodiment of any aspect herein, the formulation is a sustained release formulation the antifibrotic agent is released over an extended period of time.

In one embodiment of any aspect herein, the formulation is a sustained release formulation the antifibrotic agent is released over an extended period of least 1 day; or at least 2 days; or at least 3 days; or at least 4 days; or at least 5 days; or at least 6 days; or at least 1 week; or at least 2 weeks; or at least 3 weeks; or at least 4 weeks; or at least 5 weeks, or at least 6 weeks; or at least 8 weeks; or at least 9 weeks; at least 10 weeks; or at least 12 weeks; or at least 15 weeks; or between 1-5 days; or between 2-5 days; or between 1-2 days; or between 2-3 days; or between 3-4 days; or between 4-5 days; or between 3-10 days; or between 1-15 weeks; or between 2-10 weeks; or between 4-8 weeks; or between 8-15 weeks; or about 1 day; or about 2 days; or about 3 days; or about 4 days; or about 5 days; or about 6 days; or about 1 week; or about 2 weeks; or about 3 weeks; or about 4 weeks; or about 5 weeks; or about 6 weeks; or about 7 weeks; or about 8 weeks; or about 9 weeks; or about 10 weeks.

Treatment of Diseases or Disorders Associated with Fibrosis

In some embodiments, a formulation as described herein is administered to a subject. In some embodiments, a formulation as described herein is used to treat an organ or location on the body of a subject, a disease or indication in a subject and or using an administration route as described in Table 1 and/or Table 2.

TABLE 1

| | | |
|---|---|---|
| Administration | Sites (non-joints) | Lung, kidney, liver, heart, skin, eye; tendons, |
| routes and | | osteotendinous junctions, tendon-bone interfaces, |
| targets | | entheses, or muscle-tendon insertions, mentioning a |
| | | slew of tendons throughout the body |
| | Sites (joints) | Jaw, spine, shoulder, elbow, wrist, hand, finger, hip, |
| | | knee, ankle, foot, toe; or any other synovial or non- |
| | | synovial joint |
| | Routes of | JOINT INJECTIONS (JI): Intraarticular, |
| | administration | periarticular, intracapsular, pericapsular |
| | | NON-JOINT DENSE CONNECTIVE TISSUE |
| | | INJECTIONS (NJDCTI): intraligamentous, |
| | | periligamentous, intratendinous, peritendinous, |
| | | intramusculotendinous, perimusculotendinous, |
| | | intraosteotendinous, or periosteotendinous; |
| | | OTHER, NON-ORTHOPEDIC: intravenously, |
| | | intramuscularly, perimuscularly, subcutaneously, |
| | | intradermally, intranasally, orally, transcutaneously |
| | | (ionto/electrophoresis), mucosally, gel, cream, |
| | | ointment, lotion, drop, suppository, spray, liquid, |
| | | powder, pulmonary inhalation, ocular. |
| Indications | When to administer | During or just after a medical procedure; for patients |
| | treatment | with stiffened joint or at risk for stiffened joint |
| | | (treatment or prophylactic) |

TABLE 2

| | |
|---|---|
| General causes of | Idiopathic, injury (trauma, medical procedure e.g. |
| fibrosis | surgery), immobility for whatever reason, |
| | inflammation, or disease/medical condition |
| Diseases/conditions: | adhesive capsulitis (injury, idiopathic, post- |
| joints (admin via | surgical, post-implant) |
| joint injection) | |
| Diseases/conditions: | idiopathic pulmonary fibrosis, cystic fibrosis, |
| lung (admin via | hypertension |
| inhalation) | |
| Diseases/conditions: | hepatitis B or C, long-term alcohol abuse, non- |
| liver | alcoholic steatohepatitis, non-alcoholic fatty liver |
| | disease, Cholestasis, autoimmune hepatitis |
| | cirrhosis |
| Diseases/conditions: | chronic kidney disease, end-stage renal disease, |
| kidney | renal interstitial fibrosis |
| Diseases/conditions: | heart failure, myocardial infarction, aortic |
| heart | stenosis, hypertrophic cardiomyopathy |
| Diseases/conditions: | Crohn's disease, inflammatory bowel disease, |
| intestine | enteropathies |
| Diseases/conditions: | scleroderma, keloids, hypertrophic scars, cellulite |
| skin (admin via | |
| intradermal injection, | |
| or transdermal) | |
| Diseases/conditions: | Peyronie's disease, uterine fibroids, urethral |
| urogenital/ | strictures |
| gynecological | |
| Diseases/conditions: | Congenital Fibrosis of the Extraocular Muscles, |
| ocular | subretinal fibrosis, epiretinal fibrosis, corneal |
| | fibrosis |
| Diseases/conditions: | Dupuytren's disease, capsular contracture of |
| connective tissue, | breast, Plantar fibromatosis, |
| fascia | |
| Diseases/conditions: | Duchenne, Becker, congenital, and other |
| neuromuscular (admin | muscular dystrophies, SMA, Charcot-Marie- |
| via joint or peri-joint | Tooth, arthrogryposis, ALS, club foot, post-polio, |
| injection) | cerebral palsy (CP), stroke, traumatic brain injury |
| | (TBI), peripheral nerve injury. |

In some embodiments, a method is provided in which the method involves identifying a subject diagnosed with one or more diseases selected from the group of diseases listed in Table 1 or Table 2 and administering a formulation of the disclosure to the subject. In some embodiments, a method is provided in which the method involves identifying a subject diagnosed with one or more diseases selected from the group of diseases consisting of Duchenne Muscular Dystrophy, Becker Muscular Dystrophy, Spinal Muscular Atrophy-Type I, Spinal Muscular Atrophy-Type II, Spinal Muscular Atrophy-Type III, Spinal Muscular Atrophy-Type IV, Cerebral Palsy, Stroke, Traumatic Brain Injury and Arthrogryposis Multiplex Congenita, fibrosis of the humeroradial joint, fibrosis of the humeroulnar joint, fibrosis of the glenohumeral joint, fibrosis of the tibiofemoral joint, fibrosis of the acetabulofemoral joint, fibrosis of the talocrural joint, fibrosis of the temporomandibular joint, fibrosis of the metacarpophalangeal joint, fibrosis of the metatarsophalangeal joint, fibrosis of the peri-articular musculature and cellulite and administering to said patient a composition or formulation of the disclosure.

### Stiffened Joint

Various compositions and methods disclosed herein may be useful for treating various aspects, precursors and related disorders of joint stiffness. Joint stiffness is a significant public health issue with current treatment options providing varied and limited outcomes. Joint stiffness can affect any joint in the body, such as a shoulder joint, an elbow joint, a wrist joint, a finger joint, a hip joint, a knee joint, an ankle joint, a toe joint, the spine and the jaw A shoulder joint is often affected by joint stiffness, which is also termed a shoulder contracture, and is also known as “frozen shoulder”.

Shoulder contracture affects approximately 2% of the U.S. population, or approximately six million individuals. While women are more often affected than men, there is no known genetic or racial predilection (Robinson C. M. et al., J. Bone Joint Surg. Br. 2012, 94 (1): 1-9; Ewald A., Am. Fam. Physician 2011, 83 (4): 417-22). Shoulder contracture recovery is arduous and protracted with a significant number of patients never regaining full joint function. The condition affects both quality of life and productivity. Its predominant feature is painful, gradual loss of both active and passive glenohumeral motion resulting from progressive fibrosis of the glenohumeral joint capsule. The contracted capsule causes pain, especially when it is stretched suddenly, and produces a mechanical restraint to motion. The disease course of primary (idiopathic) shoulder contracture begins with the slow onset (over 2 to 9 months) of pain and stiffness that progressively restricts both passive and active range of motion (ROM) in the glenohumeral joint (Sharma S., Annals of the Royal College of Surgeons of England 2011 93 (5): 343-4; discussion 5-6). The pain may sharpen at night, leaving patients unable to sleep on the affected side. Subsequently, the pain generally abates over a period of 4 to 12 months, but stiffness severely restricts ROM, particularly in the external rotational plane. There is a slow improvement in ROM over a period of 2 to 4 years. Secondary shoulder contracture has a similar presentation and progression but results from a known intrinsic or extrinsic cause (Sheridan M. A. and Hannafin J. A., Orthop. Clin. North Am. 2006, 37 (4): 531-9). Secondary shoulder contracture following trauma or surgery has a 100% incidence to varying degrees after these events and requires prolonged physical therapy, with original motion not always restored.

Shoulder contracture pathology is a thickened glenohumeral joint capsule with adhesions obliterating the axillary fold. The fibrotic capsule adheres to itself and the anatomic neck of the humerus, intraarticular volume is diminished, and synovial fluid in the joint is significantly decreased (Hand G. C. et al., J. Bone Joint Surg. Br. 2007, 89 (7): 928-32). Biopsy of the capsule shows a chronic inflammatory infiltrate, an absence of synovial lining, and subsynovial fibrosis (Ozaki J. et al., J. Bone Joint Surg. Am. 1989, 71 (10): 1511-5; Wiley A. M., Arthroscopy 1991, 7 (2): 138-43; Rodeo S. A. et al., J. Orthop. Res. 1997, 15 (3): 427-36). Patient biopsy samples confirm the presence of T-cells, B-cells, synovial cells, fibroblasts and transforming myofibroblasts, along with type-I and type-III collagen (Rodeo S. A. et al., J. Orthop. Res. 1997, 15 (3): 427-36; Bunker T. D. et al., J. Bone Joint Surg. Br. 2000, 82 (5): 768-73). Gene and protein expression assays have found products related to fibrosis, inflammation, and chondrogenesis (Hagiwara Y. et al., Osteoarthritis Cartilage 2012, 20 (3): 241-9), including increased COL1A1 and COL1A3, interleukin-6, platelet-derived growth factor (PDGF), fibroblast growth factors (FGF) and inhibitors of the matrix metalloproteinases (TIMPs), as well as decreased activity of matrix metalloproteinases (MMPs). These data indicate that inflammatory changes initiate the recruitment of fibroblasts and immune cells, precipitating the fibrotic process and inappropriate deposition of collagen. Alternatively, fibrotic changes may occur first, followed by inflammation. In this case, fibrosis may result from an underlying disease process, in which cell signaling pathways governing collagen remodeling may be defective (Bunker T. D. et al., J. Bone Joint Surg. Br. 2000, 82 (5): 768-73). For example, patients treated with marimastat, a synthetic TIMP, developed shoulder contractures, and when the marimastat was stopped, the disease regressed (Hutchinson J. W. et al., J. Bone Joint Surg. Br. 1998, 80 (5): 907-8).

Shoulder contracture is considered a self-limiting disease, but recovery is protracted and arduous, with a significant number of patients never regaining full ROM. The reported outcomes of conservative therapy (i.e., physical therapy) vary considerably and are highly dependent on how they are measured (Neviaser A. S. and Neviaser R. J., J. Am. Acad. Orthop. Surg. 2011, 19 (9): 536-42). Results tend to be more favorable with subjective outcome measures than with objective outcome measures. In one study, 90% of patients treated with minimal therapy reported satisfaction with their shoulder function (Griggs S. M. et al., J. Bone Joint Surg. Am. 2000, 82-A (10): 1398-407). However, another that used objective outcomes reported residual pain in 50% of patients and motion deficit in 60% (Shaffer B. et al., J. Bone Joint Surg. Am. 1992;74 (5): 738-46). Mild to moderate symptoms can persist after 4.4 years following symptom onset of shoulder contracture. For those experiencing severe disease, such functional impairment interferes with daily activities and work-related responsibilities (Hand C. et al., Journal of Shoulder and Elbow Surgery 2008, 17 (2): 231-6). When patients do not respond to conservative management, other treatment options are available. Operative intervention in the form of manipulation under anesthesia may restore motion and decrease pain, but it has been associated with complications such as fracture, tendon rupture, and neurologic injury (Castellarin G. et al., Archives of Physical Medicine and Rehabilitation 2004, 85 (8): 1236-40; Hsu S. Y. and Chan K. M., International Orthopaedics, 1991, 15 (2): 79-83; Parker R. D. et al., Orthopedics, 1989, 12 (7): 989-90). There are reports that manipulation or capsular release do not offer reliable and consistent results (Shaffer B. et al., J. Bone Joint Surg. Am. 1992, 74 (5): 738-46; Ryans I. et al., Rheumatology 2005, 44 (4): 529-35). Accordingly, a more effective and consistent therapy for joint stiffness is needed.

Encapsulation of biologically active agents into biocompatible and biodegradable polymeric matrices prior to administration prolongs effective therapeutic levels in a patient. Hydrogels and microparticles are implantable structures. They are desirable for therapeutic delivery due to designs that are biocompatible, made of non-toxic constituents, not immunogenic or cause irritation and do not hinder the target tissue structurally or mechanically. Significantly, they can be administered locally to the area of interest. One material that is extensively used for microencapsulation and prolonged release of small molecule drugs, DNA and proteins is poly (lactic-co-glycolic) acid (PLGA). PLGA is biocompatible and releases its payload both through diffusion of out of the polymer matrix and via breakdown of the polymer matrix. The breakdown occurs through hydrolysis of PLGA, catalyzed by the body's aqueous environment, into lactic acid and glycolic acid, which are byproducts of cellular metabolism. It is considered safe for administration to humans by the United States Food and Drug Administration (Han, F. C. et al., Front. Pharmacol. 2016, 7 (185)). PLGA microparticles can be optimized for sustained drug release by adjusting the ratio of lactic acid to glycolic acid and the emulsification protocol. However, it is known to cause a foreign body response.

Techniques for encapsulation of a biologically active agent inside lactide, glycolide co-polymer microparticles are known. The production techniques generally include either the use of two solvent phases, stabilizer, and the biologically active agent dissolved or solvated into one of the phases or the use of water/oil/water (w/o/w) or water/oil (w/o) emulsions. In the first mentioned production technique, the two phases, biologically active agent and stabilizer are emulsifier and then one of the phases is removed, leaving behind a microparticle with stabilized, loaded agent. In the w/o/w fabrication technique, the initial water phase contains or does not contain the biologically active compound, is emulsified within the organic phase containing the dissolved polymeric matrix and then emulsified within the second aqueous phase. The removal of the organic phase leaves behind a microparticle containing or not containing the biologically active compound. That said the specific methods and PLGA compositions used highly depend on the encapsultant and a general procedure/composition does not exist for all encapsultants.

Another promising material for use specifically in the field of regenerative medicine and tissue maintenance as a drug delivery system are hydrogels. These hydrogels are polymeric networks capable of encapsulating biologically active agents. Hydrogels possess relevant biological properties such as biocompatibility, sheering thinning characteristics, biodegradation, and do not impact the stability or activity of the loaded biologically active agent.

One such formulation of hydrogels involves a network of low molecular weight gelators (LMWG) that act as injectable scaffolds for biomedical applications. They have tunable physiochemical and biological properties due to their supramolecular structure stemming from the self-assembly of small molecules. Specifically, LMWGs with bolaamphiphiles consisting of a N-thymine glycosylated head groups linked to a lipidic moiety via either urea or amide functions have shown to be fast-gelling with high in vivo stability and do not activate macrophages (Ramen, F. A. et al., Biomaterials. 2017, 145:72-80). Another LMWG formulation utilizes a combination of glycosyl-nucleosides and fluorocarbon chains as amphiphiles that self-assemble into highly organized structures that increases stability of hydrogel formulations (Godeau, G., et al., Tetrahedron Letters 2010, 51:1012-1015). They demonstrated numerous advantageous properties, including biocompatibility, control over structure and purity, easy handling procedure to allow for incorporation of proteins, mechanical stability and are non-toxic to cells (Godeau, G., et al., Tetrahedron Letters 2010, 51:1012-1015; Ramen, F. A. et al., Biomaterials. 2017, 145:72-80).

Another formulation of hydrogels is the use of a PEG-based hydrogel. In this formulation the PEG-based hydrogel would include polymeric PEG matrix with a biologically active agent either linked or encapsulated to the matrix. Encapsulation would occur through localization of the biological agent into the hydrogel during polymerization. Release of the agent would occur through diffusion out of the hydrogel into the tissue. In the case of a chemical bond between the agent and the hydrogel, it would be either a cleavable or non-cleavable connection. If cleavable, the linkage would be either rely upon an enzymatic or non-enyzmatic based mechanism.

Rigid contracture or fibrosis (arthrofibrosis) of the major articular joints is a severely limiting comorbidity and sequela of many neuromotor degenerative disorders. It presents as an accumulation of fibrotic collagenous tissue within the joint and manifests as a painful and longstanding restriction of joint range of motion (ROM), contributing to poor mobility and requiring home care assistance or institutionalization.

Stiffened joint may be most limiting in the shoulders, elbows, knees, hips, wrists, and ankles of patients with progressive neuromotor disorders. Degenerative disorders that may be treated by formulations and methods provided herein and that lead to arthrofibrosis and have different etiologies and include, but are not limited to, Duchenne (DMD) and Becker (BMD) muscular dystrophies, Congenital Muscular Dystrophies (CMD), Spinal Muscular Atrophy (SMA), Charcot-Marie-Tooth disease (CMT), arthrogryposis, Emery Dreifus Muscular Dystrophy (EMD), the family of slow progressive muscular dystrophies (Limb-girdle (LGMD), Fascioscapulohumeral (FSH), Congenital Myotonic (CMMD)), Amyotrophic Lateral Sclerosis (ALS), idiopathic congenital club foot, post-polio syndrome, all forms of cerebral palsy (CP), stroke, traumatic brain injury, and peripheral nerve injury (1,2). Incidence of these conditions is on the order of 1-10/100,000 population for the dystrophies and 2-3/1000 births for cerebral palsy (2). The national cost burden of management of these conditions is significant, with population-wide national costs just for managing three of these diseases estimated to be $1,023 million (ALS), $787 million (DMD), and $448 million (CMMD) (3). The CDC estimates the overall cost of care for the population of patients with cerebral palsy born in the year 2000, will exceed 11.5 billion (4). These expenses represent medical as well as non-medical costs, and account primarily for musculoskeletal care.

The lack of joint mobility caused by arthrofibrosis in patients with a neuromotor degenerative condition or neuromotor trauma, such as stroke, traumatic brain injury, and peripheral nerve injury, contributes to further muscle tone loss, muscle fibrosis and scarring, osteoporosis, secondary deformities such as spinal scoliosis and lower extremity equinus posture, and loss of skin integrity. Ultimately, arthrofibrosis results in inability to ambulate and limits activities of daily living. In the stages of disease when patients may no longer be ambulatory, joint contracture further burdens nursing care, rest positioning, sitting, and hygiene (1).

At present, prolonged physical therapy, forceful passive stretching, serial casting, and bracing are the only non-operative treatment modalities available, with or without botulism toxin supplementation to diminish muscle associated contracture (1). Surgical Interventions to improve mobility of a fibrosed joint include manipulation under anesthesia, tendon and muscle releases, and articular capsular release or resection surgeries of the involved joints (5). Manipulation of a joint under anesthesia can result in periarticular and shaft fractures, when forceful mobilization of the fibrosed joint introduces more stress to the adjacent osteoporotic bone than it can tolerate. Many patients are also poor candidates given the intubation and ventilation required for the application of a paralyzing anesthetic agent to counter muscle resistance during a manipulation or surgical release. After prolonged periods of contracture, acute surgical joint release and manipulation may also result in severe nerve and vascular stretch injuries, with inconsistent results and variable recurrence rates.

The overall health and quality of life for these patients would be greatly enhanced by an alternative non-operative treatment modality aimed at resolving joint contracture. This disclosure, in some embodiments, provides a solution and is a non-surgical office-based intra-articular injection therapy to be used in conjunction with physical therapy to release contracted joints over a two to eight-week period.

In various aspects and embodiments, the compositions and methods provided herein can be of value to a wide range of subjects. Patients with neuromotor degenerative disease are a highly managed population, requiring a lifetime of intensive and costly medical and non-medical support. The current standard of care is either conservative treatment or surgical intervention. In contrast, the compositions and methods provided herein may in some embodiments provide a therapeutic benefit with an in-office injection, eliminating surgery, and offering mobility to an immobile patient, improving their overall health and quality of life and reducing the intensity of supportive care. Caretakers, physicians, and specialists will be able to restore joint motion without performing surgery on this at-risk patient population. Patients will benefit from improved motion and require less physical therapy to maintain joint mobility. They would retain an independent ability to mobilize and perform activities of daily living for longer periods of time as their condition progresses. They would enjoy overall improved musculoskeletal health and psychosocial benefit. For the payor, the overall health care cost for the management of these conditions would decrease as surgical cost per patient would decrease in addition to the higher likelihood that a patient would be able to remain at home longer and not require institutional care for sequela of poor mobility or inability to perform adequate care and hygiene at home.

The present disclosure provides methods for treating or preventing a stiffened joint in a subject in need thereof. The methods comprise of administering to the subject an effective amount of an agent or ligand of the relaxin family receptors, a relaxin-2 variant, relaxin-2 chemically conjugated to a targeting agent, including a single-domain camelid antibody fragment, a peptide sequence, polynucleotide, or a small molecule, such that the stiffened joint or surrounding tissue area in the subject is treated.

The current methods for treating a stiffened joint include physical therapy or surgical procedures, such as manipulations and releases, which do not offer reliable or consistent results (Diercks R. L. et al., J. Shoulder Elbow Surg. 2004, 13 (5): 499-502). Physical therapy involves prolonged manipulation by a physical therapist and surgical procedures involves invasive surgical release by a surgeon, followed again by prolonged therapy. Another current method, the Ponseti method, involves serial re-casting after stretching, sometimes with surgical release of contracted tendons.

The methods of the disclosure are, in some embodiments, advantageous as compared to many currently available methods because they can be used to reliably and effectively treat a stiffened joint or tissue area, while also using a minimally invasive procedure, e.g., an intraarticular injection, which may be performed in an outpatient setting or an office. Thus, some methods of the disclosure constitute a paradigm shift in the management of a stiffened joint, e.g., a shoulder joint, that may result from fibrosis. Some methods of the disclosure involve minimally invasive procedures, e.g., an intraarticular or periarticular injection of relaxin-2, e.g., relaxin-2 encapsulated in a sustained release formulation. The intraarticular injection may be repeated as needed until the stiffened joint is successfully treated, e.g., until motion in the joint is restored and pain during motion is eliminated. Successful treatment of a stiffened joint when using some methods of the disclosure may be accomplished significantly faster and more effectively than when using the currently available methods.

Pathology of a stiffened joint, e.g., a shoulder joint, includes a thickened glenohumeral joint capsule with adhesions obliterating the axillary fold. The fibrotic capsule adheres to itself and the anatomic neck of the humerus, intraarticular volume is diminished, and synovial fluid in the joint is significantly decreased. Biopsy of the capsule shows a chronic inflammatory infiltrate, with the presence of fibroblasts and transforming myofibroblasts, along with type-I and type-III collagen. Gene and protein expression assays have found components related to fibrosis, inflammation, and chondrogenesis, including increased COL1A1 and COL1A3, interleukin-6 (IL-6), platelet-derived growth factor (PDGF), fibroblast growth factors (FGF) and TMPs, as well as decreased MMP activity. This evidence points to inflammatory changes initiating the recruitment of fibroblasts and immune cells, precipitating the fibrotic process and inappropriate deposition of excess collagen. Alternatively, it is also possible that fibrosis occurs first, followed by inflammation; fibrosis being secondary to defective cell-signaling pathways governing collagen remodeling.

Without wishing to be bound by a specific theory, it is believed that the agent e.g., relaxin, when delivered to or near a joint, e.g., via a hydrogel or particle, intraarticular injection, sustained release formulation, promotes collagen degradation, thereby altering the homeostasis of the extracellular matrix (ECM) in the synovium. This administration results in decreased joint stiffness and increased range of motion of the joint.

In one embodiment, the antifibotic agent of the disclosure is administered as a monotherapy. In one embodiment, the antifibotic agent of the disclosure is administered with at least one additional therapeutic. Exemplary additional therapeutics include, but are not limited to, an addition anti-fibrotic therapeutic or physical therapy.

In some embodiments, relaxin or an analog, a fragment or a variant thereof is co-administered with other native anti-fibrotic agents such as IFN-a, IFN-β, srli B, M3, MMP1, MMP8. Additionally, the use of other anti-fibrotic agents that target receptors other than the relaxin receptor: TGF-beta inhibitors (Esbriet, pirfenidone), tyrosine kinase inhibitors (Ofev, nintedanib). PPAR (peroxisome proliferator-activated receptors) agonists (Ianifibranor, IVA337), IL-1 inhibitors (Arcalyst, rilonacept), IL-6 inhibitors (Actmera, tocilizumab), B-cell inhibitors (rituximab), T-cell inhibitors (Orencia, abatacept), lysophosphatidic acid inhibitors (SAR100842, Sanofi), Halofunginone, d-penicillamine, colchicine, cyclosporine, TGF beta blockers, p38 MAPK blockers

### Methods for Treating a Stiffened Joint

Some aspects of the present disclosure provide methods for treating or preventing a stiffened joint. As used herein, the terms “treating”, “treat” or “treatment” refer to a beneficial or desired result including, but not limited to, alleviation or amelioration of one or more symptoms associated with a stiffened joint (e.g., pain on movement of the joint, loss of motion of the joint or loss of the range of motion of the joint); diminishing the restriction of movement resulting from a stiffened joint; stabilization (i.e., not worsening) of the joint stiffness; amelioration or palliation of the restriction of movement resulting from a stiffened joint (e.g., pain on movement of the joint, loss of motion of the joint or loss of the range of motion of the joint) whether detectable or undetectable.

In some embodiments, methods of the present disclosure result in a treatment of the stiffened joint, such that pain on movement of the joint is reduced, e.g., by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, and is preferably down to a level accepted within the range of normal for an individual who is not affected by a stiffened joint.

In some embodiments, methods of the present disclosure result in restoration of the movement, or a range of the movement, of a joint affected by joint stiffness. For example, treatment of the stiffened joint according to the methods of the disclosure may result in restoration of the movement, or a range of movement, of a joint affected by joint stiffness, to levels that are at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the levels accepted within the range of normal for an individual not affected by a stiffened joint.

In some embodiments, prevention or treatment of a stiffened joint in a subject provided by the methods of the present disclosure is accomplished without significant adverse events, without significant damage to collagenous structures or tissues in the subject, e.g., collagenous structures or tissues of the joint, such as articular cartilage of the joint. For example, methods of the present disclosure provide prevention and treatment of stiffened joint that do not disrupt architecture of the joint. Damage to collagenous structures in the body, e.g., collagenous structures of a joint, may be assessed by methods known in the art, e.g., by measuring levels of various markers in the synovial fluid, such as Cartilage Oligomeric Matrix Protein (COMP), aggrecans, collagen II, proteoglycans, MMPs and inflammatory mediators and cytokines. Imaging techniques such as MRI can also be used to visualize the joint and the cartilage architecture.

In some embodiments, when the agent (e.g., relaxin) loaded depot is administered intraarticularly, prevention or treatments of stiffened joint by the methods of the present disclosure is accomplished without significant adverse events associated with systemic administration of relaxin. In a phase III clinical trial for use of relaxin to treat systemic sclerosis, some of the patients that received a 24-week subcutaneous infusion of relaxin had declines in creatine clearance and renal adverse events; however renal physiology abnormalities are associated with systemic sclerosis and may have predisposed the affected patients to such renal events when combined with relaxin treatment (Khanna, D., et al., Arthritis and Rheumatism 2009, 60 (4): 1102-1111). When relaxin is administered intraarticularly by methods of the present disclosure, serum creatine levels, protein levels in the urine, blood cell count, hemoglobin concentration in the blood and systolic and diastolic blood pressure will be monitored during and after administration for prevention or treatment of a stiffened joint for indication of renal crisis and hypertension.

One aspect provided herein is a method, said method comprising identifying a subject diagnosed with one or more diseases selected from the group of diseases listed in Table 1 or Table 2 and administering a formulation of any one of the preceding embodiments to the subject.

Another aspect provided herein is a method, said method comprising identifying a subject diagnosed with Duchenne Muscular Dystrophy and administering to said patient a composition or formulation of any of the preceding embodiments.

Another aspect provided herein is a method, said method comprising identifying a subject diagnosed with Becker Muscular Dystrophy and administering to said patient a composition or formulation of any of the preceding embodiments.

Another aspect provided herein is a method, said method comprising identifying a subject diagnosed with Spinal Muscular Atrophy, Type I, and administering to said patient a composition or formulation of any of the preceding embodiments.

Another aspect provided herein is a method, said method comprising identifying a subject diagnosed with Spinal Muscular Atrophy, Type II, and administering to said patient a composition or formulation of any of the preceding embodiments.

Another aspect provided herein is a method, said method comprising identifying a subject diagnosed with Spinal Muscular Atrophy, Type III, and administering to said patient a composition or formulation of any of the preceding embodiments.

Another aspect provided herein is a method, said method comprising identifying a subject diagnosed with Spinal Muscular Atrophy, Type IV, and administering to said patient a composition or formulation of any of the preceding embodiments.

Another aspect provided herein is a method, said method comprising identifying a subject diagnosed with Cerebral Palsy and administering to said patient a composition or formulation of any of the preceding embodiments.

Another aspect provided herein is a method, said method comprising identifying a subject diagnosed with Arthrogryposis Multiplex Congenita and administering to said patient a composition or formulation of any of the preceding embodiments.

Another aspect provided herein is a method, said method comprising identifying a subject with fibrosis of the humeroradial joint and administering to said patient a composition or formulation of any of the preceding embodiments.

Another aspect provided herein is a method, said method comprising identifying a subject with fibrosis of the humeroulnar joint and administering to said patient a composition or formulation of any of the preceding embodiments.

Another aspect provided herein is a method, said method comprising identifying a subject with fibrosis of the glenohumeral joint and administering to said patient a composition or formulation of any of the preceding embodiments.

Another aspect provided herein is a method, said method comprising identifying a subject with fibrosis of the tibiofemoral joint and administering to said patient a composition or formulation of any of the preceding embodiments.

Another aspect provided herein is a method, said method comprising identifying a subject with fibrosis of the acetabulofemoral joint and administering to said patient a composition or formulation of any of the preceding embodiments.

Another aspect provided herein is a method, said method comprising identifying a subject with fibrosis of the talocrural joint and administering to said patient a composition or formulation of any of the preceding embodiments.

Another aspect provided herein is a method, said method comprising identifying a subject with fibrosis of the temporomandibular joint and administering to said patient a composition or formulation of any of the preceding embodiments.

Another aspect provided herein is a method, said method comprising identifying a subject with fibrosis of the metacarpophalangeal joint and administering to said patient a composition or formulation of any of the preceding embodiments.

Another aspect provided herein is a method, said method comprising identifying a subject with fibrosis of the metatarsophalangeal joint and administering to said patient a composition or formulation of any of the preceding embodiments.

Another aspect provided herein is a method, said method comprising identifying a subject with fibrosis of the peri-articular musculature and administering to said patient a composition or formulation of any of the preceding embodiments.

Another aspect provided herein is a method, said method comprising identifying a subject with cellulite and administering to said patient a composition or formulation of any of the preceding embodiments.

Another aspect provided herein is a method, said method comprising identifying a subject with interstitial lung disease and administering to said patient a composition or formulation of any of the preceding embodiments.

Another aspect provided herein is a method, said method comprising administering, to any of the preceding subjects, a composition or formulation of any of the preceding embodiments, via inhalation as an aerosol.

Another aspect provided herein is a method, said method comprising administering, to any of the preceding subjects, a composition or formulation of any of the preceding embodiments, via intra-articular injection.

Another aspect provided herein is a method, said method comprising administering, to any of the preceding subjects, a composition or formulation of any of the preceding embodiments, via intramuscular injection.

Another aspect provided herein is a method, said method comprising administering, to any of the preceding subjects, a composition or formulation of any of the preceding embodiments, via intradermal injection.

Another aspect provided herein is a method, said method comprising administering, to any of the preceding subjects, a composition or formulation of any of the preceding embodiments, via subcutaneous injection.

Another aspect provided herein is a method, said method comprising administering, to any of the preceding subjects, a composition or formulation of any of the preceding embodiments, via intracapsular injection.

Another aspect provided herein is a method, said method comprising administering, to any of the preceding subjects, a composition or formulation of any of the preceding embodiments, via pericapsular injection.

Another aspect provided herein is a method, said method comprising administering, to any of the preceding subjects, a composition or formulation of any of the preceding embodiments, via intraligamentous injection.

Another aspect provided herein is a method, said method comprising administering, to any of the preceding subjects, a composition or formulation of any of the preceding embodiments, via periligamentous injection.

Another aspect provided herein is a method, said method comprising administering, to any of the preceding subjects, a composition or formulation of any of the preceding embodiments, via intratendinous injection.

Another aspect provided herein is a method, said method comprising administering, to any of the preceding subjects, a composition or formulation of any of the preceding embodiments, via peritendinous injection.

Another aspect provided herein is a method, said method comprising administering, to any of the preceding subjects, a composition or formulation of any of the preceding embodiments, via intramusculotendinous injection.

Another aspect provided herein is a method, said method comprising administering, to any of the preceding subjects, a composition or formulation of any of the preceding embodiments, via perimusculotendinous injection.

Another aspect provided herein is a method, said method comprising administering, to any of the preceding subjects, a composition or formulation of any of the preceding embodiments, via intraosteotendinous injection.

Another aspect provided herein is a method, said method comprising administering, to any of the preceding subjects, a composition or formulation of any of the preceding embodiments, via periosteotendinous injection.

Another aspect provided herein is a method, said method comprising administering, to a subject diagnosed with Duchene's muscular dystrophy, a composition or formulation of any of the preceding embodiments, via intramuscular injection.

Another aspect provided herein is a method, said method comprising administering, to a subject diagnosed with Duchene's muscular dystrophy, a composition or formulation of any of the preceding embodiments, via intraarticular injection.

Another aspect provided herein is a method, said method comprising administering, to a subject diagnosed with Becker's muscular dystrophy, a composition or formulation of any of the preceding embodiments, via intramuscular injection.

Another aspect provided herein is a method, said method comprising administering, to a subject diagnosed with Becker's muscular dystrophy, a composition or formulation of any of the preceding embodiments, via intraarticular injection

Another aspect provided herein is a method, said method comprising administering, to a subject diagnosed with Spinal Muscular Dystrophy, a composition or formulation of any of the preceding embodiments, via intramuscular injection.

Another aspect provided herein is a method, said method comprising administering, to a subject diagnosed with Spinal Muscular Dystrophy, a composition or formulation of any of the preceding embodiments, via intraarticular injection.

Another aspect provided herein is a method, said method comprising administering, to a subject diagnosed with Arthrogryposis Multiplex Congenita, a composition or formulation of any of the preceding embodiments, via intramuscular injection.

Another aspect provided herein is a method, said method comprising administering, to a subject diagnosed with Arthrogryposis Multiplex Congenita, a composition or formulation of any of the preceding embodiments, via intraarticular injection.

Another aspect provided herein is a method, said method comprising administering, to a subject diagnosed with Cerebral Palsy, a composition or formulation of any of the preceding embodiments, via intramuscular injection.

Another aspect provided herein is a method, said method comprising administering, to a subject diagnosed with Cerebral Palsy, a composition or formulation of any of the preceding embodiments, via intraarticular injection.

Another aspect provided herein is a method, said method comprising administering, to a subject diagnosed with Stroke, a composition or formulation of any of the preceding embodiments, via intramuscular injection.

Another aspect provided herein is a method, said method comprising administering, to a subject diagnosed with Stroke, a composition or formulation of any of the preceding embodiments, via intraarticular injection.

Another aspect provided herein is a method, said method comprising administering, to a subject diagnosed with Traumatic Brain Injury, a composition or formulation of any of the preceding embodiments, via intramuscular injection.

Another aspect provided herein is a method, said method comprising administering, to a subject diagnosed with Traumatic Brain Injurt, a composition or formulation of any of the preceding embodiments, via intraarticular injection.

Another aspect provided herein is a method, said method comprising administering, to a subject diagnosed with Peripheral Nerve Injury, a composition or formulation of any of the preceding embodiments, via intramuscular injection.

Another aspect provided herein is a method, said method comprising administering, to a subject diagnosed with Peripheral Nerve Injury, a composition or formulation of any of the preceding embodiments, via intraarticular injection.

Another aspect provided herein is a method, said method comprising administering any of the preceding embodiments with sizes between 1 μm-10 μm via inhalation as an aerosol.

Another aspect provided herein is a method, said method comprising administering any of the preceding embodiments with sizes between 20 μm-100 μm via intramuscular injection.

Another aspect provided herein is a method, said method comprising administering any of the preceding embodiments with sizes between 5 μm-50 μm via intraarticular injection.

Another aspect provided herein is a method, said method comprising administering, to a subject diagnosed with interstitial lung disease any of the preceding embodiments via inhalation as an aerosol.

Another aspect provided herein is a method, said method comprising administering, to a subject diagnosed with interstitial lung disease any of the preceding embodiments via inhalation as an aerosol.

Another aspect provided herein is a method, said method comprising administering, to a subject diagnosed with interstitial lung disease any of the preceding embodiments, wherein the diameter of the microparticle is 1-10 μm, via inhalation as an aerosol.

Another aspect provided herein is a method, said method comprising administering, to a subject diagnosed with Duchene's Muscular Dystrophy any of the preceding embodiments, wherein the diameter of the microparticle is 10-30 μm, via intraarticular injection.

Another aspect provided herein is a method, said method comprising administering, to a subject diagnosed with Duchene's Muscular Dystrophy any of the preceding embodiments, wherein the diameter of the microparticle is 25-50 μm, via intraarticular injection.

Another aspect provided herein is a method, said method comprising administering, to a subject diagnosed with Duchene's Muscular Dystrophy any of the preceding embodiments, wherein the diameter of the microparticle is 10-30 μm, via intramuscular injection.

Another aspect provided herein is a method, said method comprising administering, to a subject diagnosed with Duchene's Muscular Dystrophy any of the preceding embodiments, wherein the diameter of the microparticle is 25-50 μm, via intramuscular injection.

Another aspect provided herein is a method, said method comprising administering, to a subject diagnosed with Spinal Muscular Atrophy any of the preceding embodiments, wherein the diameter of the microparticle is 10-30 μm, via intraarticular injection.

Another aspect provided herein is a method, said method comprising administering, to a subject diagnosed with Spinal Muscular Atrophy any of the preceding embodiments, wherein the diameter of the microparticle is 25-50 μm, via intraarticular injection.

Another aspect provided herein is a method, said method comprising administering, to a subject diagnosed with Spinal Muscular Atrophy any of the preceding embodiments, wherein the diameter of the microparticle is 10-30 μm, via intramuscular injection.

Another aspect provided herein is a method, said method comprising administering, to a subject diagnosed with Spinal Muscular Atrophy any of the preceding embodiments, wherein the diameter of the microparticle is 25-50 μm, via intramuscular injection.

Another aspect provided herein is a method, said method comprising administering, to a subject diagnosed with joint arthrofibrosis any of the preceding embodiments, wherein the diameter of the microparticle is 10-30 μm, via intraarticular injection.

Another aspect provided herein is a method, said method comprising administering, to a subject diagnosed with joint arthrofibrosis any of the preceding embodiments, wherein the diameter of the microparticle is 25-50 μm, via intraarticular injection.

Another aspect provided herein is a method or formulation of any of the preceding embodiments, wherein the formulation is delivered via inhalation as an aerosol.

Another aspect provided herein is a method or formulation of any of the preceding embodiments, wherein the formulation is delivered via intra-articular injection.

Another aspect provided herein is a method or formulation of any of the preceding embodiments, wherein the formulation is delivered via intramuscular injection.

In one embodiment, method or formulation of any one of the preceding embodiments wherein, the formulation is administered to the subject such that the antifibrotic agent (e.g., a relaxin) is administered to a subject at a dose between 1-2000 μg/kg body weight; or between 10-100 μg/kg body weight; or between 100-200 μg/kg body weight; or between 200-500 μg/kg body weight; or between 500-1000 μg/kg body weight; or 25-75 μg/kg body weight; or 30-70 μg/kg body weight; or 40-60 μg/kg body weight; or between 1-10 μg/kg body weight; or between 1-5 μg/kg body weight; or between 4-8 μg/kg body weight; or about 2 μg/kg body weight; or about 5 μg/kg body weight; or about 10 μg/kg body weight; or about 20 μg/kg body weight; or about 25 μg/kg body weight; or about 30 μg/kg body weight; or about 35 μg/kg body weight; or about 40 μg/kg body weight; or about 45 μg/kg body weight; or about 50 μg/kg body weight; or about 55 μg/kg body weight; or about 60 μg/kg body weight; or about 65 μg/kg body weight; or about 70 μg/kg body weight; or about 75 μg/kg body weight; or about 100 μg/kg body weight; or about 200 μg/kg body weight; or about 500 μg/kg body weight.

### Administration

In some embodiments, methods of the disclosure comprise administering an agent e.g., relaxin or an analog, a fragment or a variant thereof to a subject using a depot. The terms “administer”, “administering” or “administration” include any method of delivery of agent into the subject's system or to a particular region in or on the subject. For example, relaxin or agent loaded depot may be administered intravenously, intramuscularly, subcutaneously, intradermally, intranasally, orally, transcutaneously, mucosally, intraarticularly, periarticularly, intracapsularly, pericapsularly, intratendinously, peritendinously, intraligamentously, periligamentously, by pulmonary inhalation or by ocular specific routes of administration. Administering the agent loaded depot can be performed by a number of people working in concert and can include, for example, prescribing relaxin or an analog, a fragment or a variant thereof to be administered to a subject via a depot and/or providing instructions, directly or through another, to take the relaxin or an analog, a fragment or a variant thereof, either by self-delivery via a depot, e.g., as by oral delivery, subcutaneous delivery, intravenous delivery through a central line, etc., or for delivery by a trained professional, e.g., intra-articular delivery, intravenous delivery, intramuscular delivery, intratumoral delivery, etc.

In a preferred embodiment, the agent e.g., relaxin or an analog, a fragment or a variant thereof is administered locally, e.g., directly to or into a joint of a subject using a depot. Local administration of the agent (e.g., relaxin) loaded depot by an intraarticular injection or by topical application to the joint, or in the tissue surrounding the joint is advantageous because it allows delivery of a smaller dose of the agent to the subject and avoids the side-effects associated with systemic delivery, such as back pain and joint pain.

In one embodiment, the agent e.g., relaxin loaded depot is administered to the subject by an intraarticular injection. In one embodiment, the agent e.g., relaxin loaded depot is administered to the subject by an intraarticular, periarticular, intracapsular, pericapsular, intraligamentous, periligamentous, intratendinous, peritendinous, intraosteotendinous, or periosteotendinous injection (collectively “joint injections”), or combination thereof. In one embodiment, the agent e.g., relaxin loaded depot is administered to the subject via a single joint injection. In one embodiment, the agent e.g., relaxin loaded depot is administered to the subject via multiple joint injections. The multiple joint injections of the agent e.g., relaxin loaded depot may be administered to a subject at regularly spaced time intervals, e.g., every day, every 2 days, every 3 days, every 4 days, every 5 days, every 6 days, every 7 days, every 8 days, every 9 days, every 10 days, every 11 days, every 12 days every 13 days or every 14 days. A course of treatment consisting of multiple joint injections of agent e.g., relaxin loaded depot may be repeated.

In one embodiment, the agent is administered to or near tendons, osteotendinous junctions, tendon-bone interfaces, entheses, or muscle-tendon insertions. Such tissues may be selected from the following tendinous tissues, among others:
Shoulder
   Teres Minor Tendons (Rotator Cuff) Infraspinatus Tendons Supraspinatus Tendons Subscapularis Tendons
Elbow/Forearm
   Deltoid Tendons Biceps Tendons Triceps Tendons Brachioradialis Tendons Extensor Carpi Radialis Brevis Tendons Extensor Carpi Radialis Longus Tendons Supinator Tendons
Wrist
   Flexor Carpi Radialis Tendons Flexor Carpi Ulnaris Tendons Extensor Capri Radialis Tendons Extensor Carpi Radialis Brevis Tendons
Hip/Groin
   Iliopsoas Tendons Obturator Internus Tendons Adductor Longus, Brevis, and Magnus Tendons Gluteus Maximus and Gluteus Medius Tendons Iliotibial Band
Knee
   Quadriceps Tendons Patellar Tendons Hamstring Tendons Sartorius Tendons.
Ankle
   Gastrocnemius Tendons Achilles Tendons Soleus Tendons Tibialis Anterior Tendons Peroneus Longus Tendons
Hand (Fingers)
   Flexor Digitorum Longus Tendons Interosseus Tendons Flexor Digitorum Profundus Tendons Abductor Digiti Minimi Tendons
Hand (Thumb)
   Opponens Pollicis Tendons Flexor Pollicis Tendons Extensor and Abductor Pollicis Tendons
Foot (Toes)
   Flexor Hallucis Longus Tendons Flexor Digitorum Brevis Tendons Lumbrical Tendons Abductor Hallucis Tendons Flexor Digitorum Longus Tendons Abductor Digiti Minimi Tendons Plantar Fasciitis.
Back
   Multifidus Tendons Quadratus Lumborum Tendons Longissmus Thoracis Tendons Iliocostalis Tendons Spinalis Thoracis Tendons Psoas Major Tendons

The joint injection of the agent e.g., relaxin loaded depot may be accomplished by using a syringe with a needle suited for a joint injection. A needle suitable for an joint injection may be selected from the group consisting of a 30G needle, a 29G needle, a 28G needle, a 27G needle, a 26 sG needle, a 26G needle, a 25.5G needle, a 25 sG needle, a 25G needle, a 24.5G needle, a 24G needle, a 23.5G needle, a 23 sG needle, a 23G needle, a 22.5G needle, a 22 sG needle, a 22G needle, a 21.5G needle, a 21G needle, a 20.5G needle, a 20G needle, a 19.5G needle, a 19G needle, a 18.5G needle and an 18G needle. In a specific embodiment, the agent e.g., relaxin loaded depot is administered via a 21G needle.

The inventions illustratively described herein may be practiced in the absence of any element or elements, limitation or limitations which is not specifically disclosed herein. Thus, for example, the terms “comprising”, “including,” containing”, etc. shall be read expansively and without limitation. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present inventions have been specifically disclosed by preferred methods, embodiments and optional features, modification and variation of the concepts herein disclosed may be resorted to by those skilled in the art, and that such modifications and variations are considered to be within the scope of the inventions as defined by the embodiments and elsewhere in the invention. In the case of conflict, the specification, including definitions, will control.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

Certain aspects and embodiments of the invention and inventions have been described broadly and generically herein. Each of the narrower species and subgeneric groupings falling within the generic invention also form part of some aspects and embodiments of inventions contemplated herein. This includes the generic description of inventions with a proviso or negative limitation removing any subject matter from the genus, regardless of whether or not the excised material is specifically recited herein.

For convenience, the meaning of some terms and phrases used in the invention, examples, and appended claims, are provided. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

The term “about” as used herein means in quantitative terms plus or minus 10%. For example, “About 3%” would encompass 2.7-3.3% and “About 10%” would encompass 9-11%”. Moreover, where “about” is used herein in conjunction with a quantitative term it is understood that in addition to the value plus or minus 10%, the exact value of the quantitative term is also contemplated and described—for example, the term “about 3%” expressly contemplates, describes and includes exactly 3%.

The term “biotherapeutic ligand” as used herein refers to a molecule or arrangement of molecules or protein or nucleic acid with an intended therapeutic effect through interaction with a biological target. The term “ligand” does not restrict the molecule or arrangement of molecules to only those which specifically bind or localize to a receptor, enzyme, nucleic acid, or other biological target. The term “ligand” encompasses a molecule or an arrangement of molecules which may transiently or permanently interact or bind with a biological target. In some embodiments, a biotherapeutic ligand is an antifibrotic agent. In some embodiments, a biotherapeutic is agonist of the receptor RXFP1. In some embodiments, a biotherapeutic ligand is a relaxin.

The term “target” or “biological target” as used herein refers to a biological substance, compound, macromolecule, protein, nucleic acid, or molecule, which after activation or inhibition has a specific and intended downstream therapeutic effect. The term “target” encompasses cell surface receptors such as g protein-coupled receptors, tyrosine kinase transmembrane receptors, hormone receptors, proteases, diesterases, phosodiesterases, chemokine receptors, and ligand-gated ion channels. The term “target” or “biological target” may also encompass enzymes, voltage gated ion channels, structural proteins, enzymatic proteins, nucleic acids, transporters, or another ligand.

The term “potentiator” as used herein refers to a molecule or arrangement of molecules or protein or nucleic acid with a biological effect whereby the gene expression, protein stability, half-life, degradation resistance, translational efficiency, cell surface internalization, or proteolytic processing of a target is altered. The biological effect of the potentiator may be the linked or unlinked to the aforementioned modulation of the ligand target.

The term “carrier” as used herein refers to a formulation which allows for the sustained release of the potentiator and ligand over a given time period. The term “carrier” may also refer to a formulation that allows for simultaneous administration of potentiator and ligand, with or without an alteration to the release kinetics of the potentiator or ligand from the carrier. The term “carrier” may also refer to an excipient, diluent, cream, lotion, gel, particle, film, hydrogel, or carrier solution.

In addition to the aspects and embodiments disclosed in the instant disclosure, the following embodiments are specifically contemplated:

1. A method, wherein said method comprises administering to a subject a potentiator and a biotherapeutic ligand.

2. A method, wherein said method comprises administering to a subject a potentiator and a biotherapeutic ligand; wherein the potentiator upregulates the expression of a receptor of the biotherapeutic ligand in the subject.

3. A method, wherein said method comprises administering to a subject a potentiator and a biotherapeutic ligand; wherein the potentiator upregulates the expression of a receptor of the biotherapeutic ligand in the subject.

4. A method, wherein said method comprises administering to a subject a potentiator and a biotherapeutic ligand; wherein the potentiator is a corticosteroid.

5. A method, wherein said method comprises administering to a subject a potentiator and a biotherapeutic ligand; wherein the biotherapeutic ligand is a relaxin.

6. A method, wherein said method comprises administering to a subject a potentiator and a biotherapeutic ligand; wherein the potentiator is a corticosteroid and the biotherapeutic ligand is a relaxin.

7. A method of treating a subject, said method comprising identifying a subject diagnosed with one or more diseases selected from the group of diseases listed in Table 1 or Table 2 and administering to said subject a potentiator and a biotherapeutic ligand.

8. A method of treating a subject, said method comprising identifying a subject diagnosed with one or more diseases selected from the group of diseases listed in Table 1 or Table 2 and administering to said subject a potentiator and a biotherapeutic ligand; wherein the potentiator is a corticosteroid.

9. A method of treating a subject, said method comprising identifying a subject diagnosed with one or more diseases selected from the group of diseases listed in Table 1 or Table 2 and administering to said subject a potentiator and a biotherapeutic ligand; wherein the biotherapeutic ligand is a relaxin.

10. A method of treating a subject, said method comprising identifying a subject diagnosed with one or more diseases selected from the group of diseases listed in Table 1 or Table 2 and administering to said subject a potentiator and a biotherapeutic ligand; wherein the potentiator is a corticosteroid and the biotherapeutic ligand is a relaxin.

11. A therapeutic regimen, wherein said therapeutic regimen comprises a potentiator and a biotherapeutic ligand.

12. A therapeutic regimen, wherein said therapeutic regimen comprises a potentiator and a biotherapeutic ligand and wherein said potentiator is a corticosteroid.

13. A therapeutic regimen, wherein said therapeutic regimen comprises a potentiator and a biotherapeutic ligand and wherein said biotherapeutic ligand is a relaxin.

14. A therapeutic regimen, wherein said therapeutic regimen comprises a potentiator and a biotherapeutic ligand and wherein said potentiator is a corticosteroid and said biotherapeutic ligand is a relaxin.

15. A pharmaceutical composition, wherein said pharmaceutical composition comprises a potentiator and a biotherapeutic ligand.

16. A pharmaceutical composition, wherein said pharmaceutical composition comprises a potentiator and a biotherapeutic ligand and wherein said potentiator is a corticosteroid.

17. A pharmaceutical composition, wherein said pharmaceutical composition comprises a potentiator and a biotherapeutic ligand and wherein said biotherapeutic ligand is a relaxin.

18. A pharmaceutical composition, wherein said pharmaceutical composition comprises a potentiator and a biotherapeutic ligand and wherein said potentiator is a corticosteroid and said biotherapeutic ligand is a relaxin.

19. A microparticle pharmaceutical composition, wherein said microparticles comprise a potentiator and a biotherapeutic ligand.

20. A microparticle pharmaceutical composition, wherein said microparticles comprise a potentiator and a biotherapeutic ligand and wherein said potentiator is a corticosteroid.

21. A microparticle pharmaceutical composition, wherein said microparticles comprise a potentiator and a biotherapeutic ligand and wherein said biotherapeutic ligand is a relaxin.

22. A microparticle pharmaceutical composition, wherein said microparticles comprise a potentiator and a biotherapeutic ligand and wherein said potentiator is a corticosteroid and said biotherapeutic ligand is a relaxin.

23. The method or composition of any one of the preceding paragraphs wherein, the formulation is administered to the subject such that the antifibrotic agent (e.g., a relaxin) is administered to a subject at a dose between 1-2000 μg/kg body weight; or between 10-100 μg/kg body weight; or between 100-200 μg/kg body weight; or between 200-500 μg/kg body weight; or between 500-1000 μg/kg body weight; or 25-75 μg/kg body weight; or 30-70 μg/kg body weight; or 40-60 μg/kg body weight; or between 1-10 μg/kg body weight; or between 1-5 μg/kg body weight; or between 4-8 μg/kg body weight; or about 2 μg/kg body weight; or about 5 μg/kg body weight; or about 10 μg/kg body weight; or about 20 μg/kg body weight; or about 25 μg/kg body weight; or about 30 μg/kg body weight; or about 35 μg/kg body weight; or about 40 μg/kg body weight; or about 45 μg/kg body weight; or about 50 μg/kg body weight; or about 55 μg/kg body weight; or about 60 μg/kg body weight; or about 65 μg/kg body weight; or about 70 μg/kg body weight; or about 75 μg/kg body weight; or about 100 μg/kg body weight; or about 200 μg/kg body weight; or about 500 μg/kg body weight.

24. A method, said method comprising identifying a subject diagnosed with one or more diseases selected from the group of diseases listed in Table 1 or Table 2 and administering a composition or method of any one of the preceding paragraphs to the subject.

25. A method, said method comprising identifying a subject diagnosed with Duchenne Muscular Dystrophy and administering to said patient a composition or method of any of the preceding paragraphs.

26. A method, said method comprising identifying a subject diagnosed with Becker Muscular Dystrophy and administering to said patient a composition or method of any of the preceding paragraphs.

27. A method, said method comprising identifying a subject diagnosed with Spinal Muscular Atrophy, Type I, and administering to said patient a composition or method of any of the preceding paragraphs.

28. A method, said method comprising identifying a subject diagnosed with Spinal Muscular Atrophy, Type II, and administering to said patient a composition or method of any of the preceding paragraphs.

29. A method, said method comprising identifying a subject diagnosed with Spinal Muscular Atrophy, Type III, and administering to said patient a composition or method of any of the preceding paragraphs.

30. A method, said method comprising identifying a subject diagnosed with Spinal Muscular Atrophy, Type IV, and administering to said patient a composition or method of any of the preceding paragraphs.

31. A method, said method comprising identifying a subject diagnosed with Cerebral Palsy and administering to said patient a composition or method of any of the preceding paragraphs.

32. A method, said method comprising identifying a subject diagnosed with Arthrogryposis Multiplex Congenita and administering to said patient a composition or method of any of the preceding paragraphs.

33. A method, said method comprising identifying a subject with fibrosis of the humeroradial joint and administering to said patient a composition or method of any of the preceding paragraphs.

34. A method, said method comprising identifying a subject with fibrosis of the humeroulnar joint and administering to said patient a composition or method of any of the preceding paragraphs.

35. A method, said method comprising identifying a subject with fibrosis of the glenohumeral joint and administering to said patient a composition or method of any of the preceding paragraphs.

36. A method, said method comprising identifying a subject with fibrosis of the tibiofemoral joint and administering to said patient a composition or method of any of the preceding paragraphs.

37. A method, said method comprising identifying a subject with fibrosis of the acetabulofemoral joint and administering to said patient a composition or method of any of the preceding paragraphs.

38. A method, said method comprising identifying a subject with fibrosis of the talocrural joint and administering to said patient a composition or method of any of the preceding paragraphs.

39. A method, said method comprising identifying a subject with fibrosis of the temporomandibular joint and administering to said patient a composition or method of any of the preceding paragraphs.

40. A method, said method comprising identifying a subject with fibrosis of the metacarpophalangeal joint and administering to said patient a composition or method of any of the preceding paragraphs.

41. A method, said method comprising identifying a subject with fibrosis of the metatarsophalangeal joint and administering to said patient a composition or method of any of the preceding paragraphs.

42. A method, said method comprising identifying a subject with fibrosis of the peri-articular musculature and administering to said patient a composition or method of any of the preceding paragraphs.

43. A method, said method comprising identifying a subject with cellulite and administering to said patient a composition or method of any of the preceding paragraphs.

44. A method, said method comprising identifying a subject with interstitial lung disease and administering to said patient a composition or method of any of the preceding paragraphs.

### EXAMPLES

Example 1: Potentiation of Relaxin for Treatment of Scleroderma. There is evidence that in scleroderma patients, RXFP1 expression changes were hypothesized to lead to the failure of relaxin as a treatment in previous clinical trials (Corallo C., et al., N. Clin Exp Rheumatol. 2019 July-August; 37 Suppl 119 (4): 69-75). To overcome these barriers, dexamethasone is used as a potentiator of RXFP1 expression and thus to amplify the antifibrotic effect of relaxin. The patient receives a 100 μl subcutaneous injections of a relaxin loaded hydrogel in the center of each square inch of sclerotic tissue; total dose per treatment location not to exceed 250 μg/kg. The patient is instructed to apply a topical lotion containing dexamethasone three times daily for the following 14 days. The patient is monitored for improvement in fibroses. Tissue mobility and elasticity as well as Rodnan skin score is assessed overtime to determine severity of scleroderma after treatment.

Example 2: Potentiation of relaxin for treatment of musculoskeletal fibrosis. Microparticles, comprised of PLGA (molar ratio 50:50 lactide:glycolide, M. W. 50,000-75,000 daltons, carboxylic acid terminated), and loaded at 1% relaxin weight/weight and 10% w/w dexamethasone (RLX-DEX MPs), are administered to a patient diagnosed with shoulder adhesive capsulitis. Prior to injection, RLX-DEX MPs are resuspended in a sterile, isotonic carboxymethylcellulose diluent to a total volume such that the final dose is 50 μg/kg body weight. Administration is in the form of 1 ml intraarticular injection using a 25G needle. As the dexamethasone releases from the RLX-DEX MPs, RXFP1 is upregulated in surrounding tissue and subsequently magnifies the antifibrotic activity of the released relaxin. Following injection, the patient is monitored for changes in joint range of motion, (e.g. internal rotation, external rotation, pronation, supination, flexion, extension, abduction, and adduction) patient reported pain, mobility, patient reported autonomy, and patient reported quality of life.

Example 3: potentiation of relaxin by dexamethasone for treatment of acute heart failure. Previous clinical exploration of relaxin for the treatment of acute heart failure (AHF) failed to demonstrate efficacy in primary endpoints in a multicenter phase III clinical trial (Metra et al., N Engl J Med 2019; 381:716-726). To potentiate the angiogenic and hemodynamic effects of relaxin, acute heart failure patients simultaneously given dexamethasone and relaxin, administered via continuous intravenous infusion at 5 μg/kg/day and 30 μg/kg/day respectively for 48 hours. Patients are continually monitored for respiratory rate, blood pressure, heart rate, and infarction reoccurrence. All-cause mortality is assessed 180 days after treatment to determine treatment efficacy at prevention of future infarcts.

Example 4: potentiation of relaxin by dexamethasone for the treatment of hepatic fibrosis. Liposomes, 90-110 nm in diameter and comprising an aqueous core loaded with relaxin at 5% w/w and encapsulated by a lipid bilayer with dexamethasone loaded into the lipid layer of the shell at 5% w/w are administered, via intravenous injection, to a patient with hepatic fibrosis due to non-alcoholic fatty liver disease. The natural tendency of nanoparticles to localize to the liver will allow for targeted delivery of relaxin and dexamethasone to hepatocytes. Patients are primarily monitored for changes in histological NASH activity score, as well as changes in insulin sensitivity and serum alanine aminotransferase levels.

Example 5: The potentiation of relaxin by dexamethasone for the treatment of pulmonary fibrosis. There is evidence that suggests a lack of sufficient RXFP1 expression in the pulmonary tissue of patients with pulmonary fibrosis (Tan J, et al. Am J Respir Crit Care Med. 2016 Dec. 1;194 (11): 1392-1402) (Bahudhanapati H, et al.] Biol Chem. 2019 Mar. 29;294 (13): 5008-5022). Dexamethasone is administered via nebulization, 100 μg twice daily, for three days. After 72 hours of dexamethasone alone nebulization therapy, the patient is administered nebulized relaxin at 10 μg/kg, twice daily alongside the continued nebulization of 100 μg dexamethasone. Combined dexamethasone-relaxin therapy is continued for five days. Following administration, the patient is monitored for decreases in pathological hallmarks of fibrosis via CT scan, as well as for increased forced vital capacity, and decrease in respiratory distress symptoms.

Example 6. The potentiation of relaxin as an antifibrotic by dexamethasone. The present invention provides compositions of matter and methods for treating fibrotic diseases including stiffened fibrotic joint capsules, muscle fibrosis (i.e. contractures due to Duchene's muscular dystrophy, spinal muscular atrophy, cerebral palsy, traumatic brain injury, immobilization) lung fibrosis (i.e. idiopathic pulmonary fibrosis, cystic fibrosis, hypertension), liver fibrosis (i.e. hepatitis B or C, long-term alcohol abuse, nonalcoholic steatohepatitis, non-alcoholic fatty liver disease, Cholestasis, autoimmune hepatitis cirrhosis), kidney fibrosis (i.e. chronic kidney disease, end-stage renal disease, renal interstitial fibrosis), heart disease (i.e. heart failure, myocardial infarction, aortic stenosis, hypertrophic cardiomyopathy), intestinal disease (i.e. Crohn's disease, inflammatory bowel disease, enteropathies, and other intestinal fibrosis), skin conditions (i.e. scleroderma, keloids, hypertrophic scars, cellulite), urogenital and gynecological conditions (Peyronie's disease, uterine fibroids) and ocular diseases (i.e. Congenital Fibrosis of the Extraocular Muscles, subretinal fibrosis, epiretinal fibrosis, corneal fibrosis) in a subject by administering a carrier containing a potentiator (i.e. cortiocosteroid, oligonucleotide) with the purpose of increasing relaxin family peptide receptors (RXFP1, RXFP2, RXFP3, RXFP4) expression in the tissue of interest, as well as a binding agent for relaxin family peptide receptors (RXFP1, RXFP2, RXFP3, RXFP4). The agent will be the native ligand of the receptor, relaxin-2, or a relaxin-2 variant. The carrier is an object with a volume of at least 0.1 μm3 and is comprised of one or more polymers or self-assembled small molecules that delivers the minimally effective clinical dose over several weeks to several months. The potentiator and relaxin-loaded carrier may be administered intravenously, intramuscularly, subcutaneously, intradermally, intranasally, orally, transcutaneously, mucosally, intraarticularly, periarticularly, intracapsularly, pericapsularly, intratendinously, peritendinously, intraligamentously, periligamentously, by pulmonary inhalation or by ocular specific routes of administration as a sustained release formulation and may be provided as a single injection or a series of injection.

### Example 7. Synthesis and Characterization of RLX and Dexamethasone DEX) Microparticles

For this study, poly (lactide-co-glycolide) (PLGA) was selected to encapsulate RLX and DEX. DEX-loaded PLGA microparticles (DEX MPs) were synthesized following a modification of a published oil/water emulsion procedure (Hickey, T. et al., Biomaterials 23, 1649-1656 (2002)) at 20 wt %. RLX-loaded PLGA microparticles (RLX MPs) were optimized using a water/oil/water double emulsion method (Igartua, M. et al. Int. J. Pharm. 169, 45-54 (1998)) with RLX loaded at 0.127 wt % as determined by ELISA.

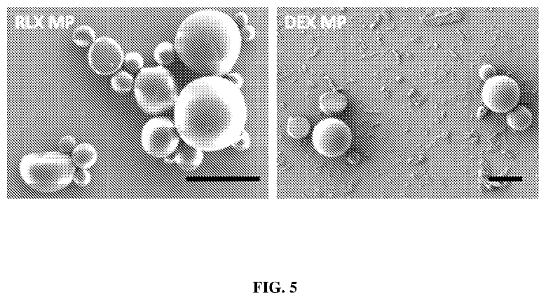

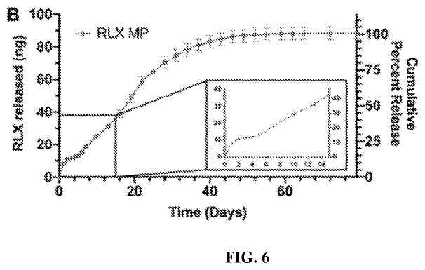

Scanning electron microscopy (SEM) shows RLX MPs (left) and DEX MPs (right) to be spherical (FIG. 5) with diameters of ˜7.7 μm and ˜10.5 μm, respectively, and low polydispersities as measured by DLS and SEM image analysis. In an in vitro release study, PLGA microparticles demonstrate a quasilinear release of RLX over the first 4 weeks that tapers around 40 days (FIG. 6) as the particles degrade under simulated physiological conditions.

### Example 8. Restoration of ROM and Reduction of Shoulder Contracture after a Single Administration of RLX-Loaded Microparticles (i.e., Single Intraarticular Injection RLX MPs, SIA RLX MPs)

To determine the efficacy of a sustained release intraarticular delivery system for RLX without DEX, a previously validated in vivo atraumatic murine model of shoulder arthrofibrosis was used to examine the ability of RLX to rescue ROM of the fibrotic shoulder over 8 weeks of treatment via live, longitudinal biomechanical measurement and terminal histological assessment.

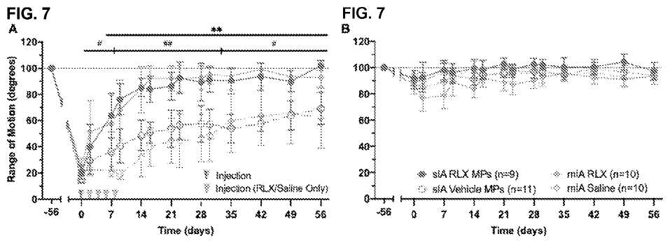

Individual baseline torque measurements at 100° internal and 60° external rotation of the healthy shoulders were used to normalize measurements made between animals. Subsequent shoulder ROM was measured in the study as the angle of rotation achieved upon reaching these baseline torque levels. The animals (Sprague-Dawley rats, 250 g) were randomly assigned to one of four treatment groups: multiple IA injections of free RLX (mIA RLX: 5×2 μg/kg, 50 μL), multiple IA injections of saline (mIA Saline, 5×50 μL), single intraarticular injection of RLX MPs (SIA RLX MPs: 1×10 μg/kg, 50 μL), or unloaded microparticles (sIA Vehicle MPs: 1×10 μg equivalent/kg, 50 μL). Longitudinal measurements of the internal ROM for each treatment group shows sIA RLX MPs achieved and maintained a statistically significant difference from mIA Saline and sIA Vehicle MP control groups (final ROM significance, p=0.03, 0.04). Significantly, sIA RLX MPs are also as effective at recovering ROM as mIA RLX (p>0.05 across all timepoints) (FIG. 7A). Final internal ROM measurements for each group respectively were 92.87°±6.84°, 62.30°±20.11°, 102.04°±3.18°, and 69.30°±10.89° (mean±95% CI). Control measurements on the contralateral shoulder for all treatment groups demonstrate that longitudinal measurement of ROM does not impact the healthy joint (FIG. 7B).

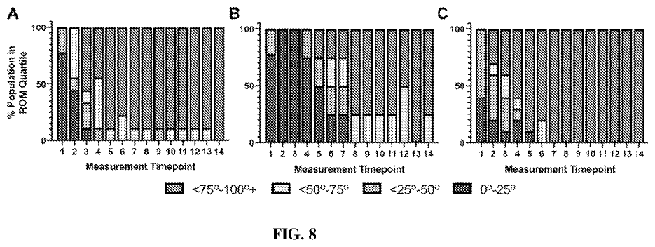

A pilot dosing study for the RLX MPs in which the microparticle composition was maintained and the total microparticle mass administered was altered to vary RLX dose was also performed. FIG. 8 shows the percentage of the rats in each treatment group with an internal ROM measured within each quartile (0°-25°, 25°-50°, 50°-75°) 75°-100°. Single intraarticular injection of low-dose RLX-loaded PLGA microparticles (one-third the dose of sIA RLX MPs, SIA RLX^{1/3} MPs, 3.33 μg/kg, 1×50 μL) did not rescue internal ROM as efficiently as sIA RLX MPs (10 μg/kg. 1×50 μL). This suggests that 3.33 μg/kg is below the MED for RLX alone and can be used for future assessment of in vivo potentiation of RLX by DEX.

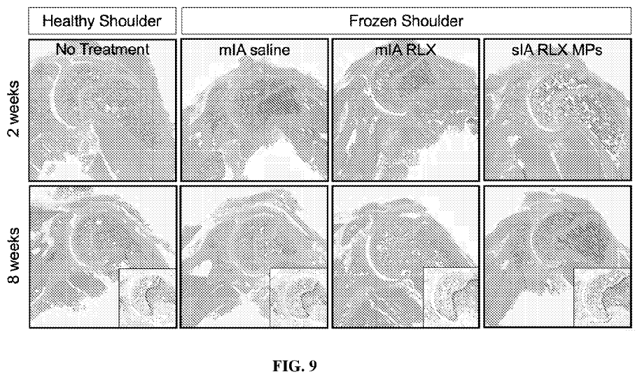

ROM recovery after sIA RLX MPs was corroborated by mid-point (2 weeks post-treatment) and end-point (8 weeks post-treatment) histochemical analyses of the humeral head and glenohumeral joint space of contracted shoulders with different treatments (FIG. 9). H&E staining of the healthy joint shows a well-delineated separation between the capsule and the articular surface of the humeral head, and the synovial membrane and articular cartilage present with normal cellular organization (FIG. 9; Healthy shoulder). There was no local adverse effects after treatment of RLX in a healthy joint. The joint of contracted shoulders receiving mIA injections of saline had severely reduced glenohumeral joint space with the capsule tightly surrounding the humeral head and evidence of capsular adhesions (FIG. 9; Frozen shoulder+mIA Saline). The synovial membrane and cartilage nuclei failed to maintain the expected tangential orientation to the humeral head within the superficial zone (tangential zone), and some surrounding fibrotic cells showed orthogonal directionality from the expected surface contour. These results are similar to a previously published shoulder contracture untreated control. Blessing et al., Proc. Natl. Acad. Sci. 116, 12183-12192 (2019).

In contrast to the saline treated control, the synovial membrane and articular cartilage surfaces remained separated in the mIA RLX group with proper cellular organization, analogous to the healthy untreated shoulder (FIG. 9; Frozen shoulder+mIA RLX). However, the mIA RLX group had less surrounding connective tissue when compared to the healthy shoulder. In a contracted shoulder treated with sIA RLX MPs, the synovial membrane and articular cartilage surfaces exhibit proper cellular organization with a well-delineated separation between the capsule and the articular surface on the humeral head (FIG. 9; Frozen shoulder+SIA RLX MP). Masson's trichrome staining for collagen performed at both time points also showed reduction of fibrotic collagenous structures in the shoulder joint with mIA RLX and RLX MPs (data not shown). Safranin-O staining showed preservation of cartilage architecture in all treated shoulders after 8 weeks compared to a healthy shoulder, demonstrating that RLX treatment does not cause cartilage damage (FIG. 8, inset).

### Example 9. Administration of RLX MPs (1×10 μg/Kg, 50 μL; sIA RLX MPs) Directly into the Glenohumeral Synovial Joint in Healthy Sprague-Dawley Rats (N=5, Female) Via an Intraarticular (IA) Injection

Using an RLX-specific ELISA, we measured serum RLX levels at 1, 4, and 24 hours post-administration. All serum samples were below the detection limit of the ligand binding assay (<5 pg/mL).

### Example 10. Synthesis of Polymers and Preparation/Characterization of Microparticles

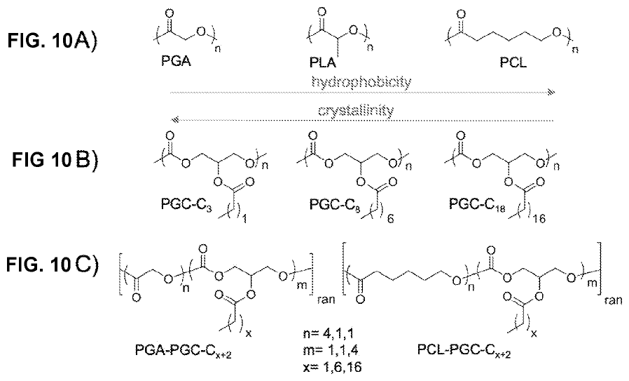

A small library of polymers and their resulting microparticles are prepared following the methods described above. For all polymers, prepare 10, 30, and 60 kDa molecular weight variants with polydispersity values less than 1.3 are prepared. The polymers are characterized via ¹H, ¹³C NMR, GPC, and DSC, as well as contact angle measurements of the polymer cast films. PGA, PLA, and PCL (FIG. 10A), and poly (glycerol monoalkylate carbonate) s possessing C3, C8 or C18 chains (FIG. 10B), as well as co-polymers of the poly (glycerol monoalkylate carbonate) s and PGA or PCL (FIG. 10C) are used to assess the effects of polymer crystallinity, hydrophobicity, and molecular weight on microparticle formulation. Microparticles with 5, 10, and 25 μm diameters are prepared using the above polymers via w/o/w double-emulsion and their size will be characterized by DLS, laser diffraction, and SEM. RLX loading will vary from 0.001 to 0.5 wt %; DEX loading will vary from 0.1 to 25% wt %.

### Example 11. RLX & DEX Release Studies

The quantity and rate of RLX or DEX release from the polymeric MPs are determined by placing a known quantity of the sample in dialysis tubing (MWCO 100 kDa) at 37° C. The RLX concentration in the surrounding aqueous solution (Dulbecco's Modified Eagle Media, with 0.4% hyaluronic acid, 10% fetal bovine serum, and 0.025% porcine esterase to simulate synovial fluid) will be measured via ELISA (R&D Systems). DEX concentration is determined by HPLC against a USP standard. Release kinetics will be modeled with the Korsmeyer-Peppas equation to quantitatively compare release constants.

### Example 12. Maintenance of Biological Activity after Encapsulation

RLX MPs are assayed in vitro for their ability to elicit biological activity and deliver a prolonged therapeutic effect. Using the assays developed in the preliminary data section, human FLS and CHON-001, as well as murine NIH3T3 and H4 cells, are cultured below a transwell insert containing RLX MPs. cAMP and collagen levels are measured at various time points (1-24 hours and 1-5 days, respectively) to determine the bioactivity of released RLX. Extended release studies are conducted by transferring the transwell insert above freshly plated cells every week for 6 weeks. To corroborate activity, HEK293 cells expressing RXFP1 and luciferase under a cAMP responsive element promoter (CRE-Luc) are treated with RLX MP-conditioned media and assessed for total luciferase activity as a proxy of cAMP levels resulting from RLX-RXFP1 activation. Structural integrity of released DEX will be confirmed via LC-MS and HPLC. The ability of DEX released from the MPs to regulate RXFP1 gene and protein expression is assessed using the cell lines listed above and analyzed via qPCR and western blot. Finally, cells will be treated with a blend of RLX MPs and DEX MPs, utilizing the determined release kinetics to provide the optimal bioactive dose over 24 hours, and assessed for RXFP1 and collagen levels to determine DEX's potentiation of RLX antifibrotic activity via RXFP1 modulation.

### Example 13. Microparticle Characteristics

The degradation characteristics of RLX MPs and DEX MPs will be determined at 1 d, 3 d, 7 d, 14 d, 21 d, 28 d, 42 d, 56 d, and 70d in the synovial fluid mimetic as described above. Release buffer samples will be analyzed by SEM and GPC to determine size and polymer molecular weight.

Clinically, a 21G or smaller needle is optimal for intraarticular injection. Therefore, the microparticle suspension developed are able to flow freely through a 21G needle, e.g., at a rate of 1 mL of particles injected in <15 seconds. If the injection rate is too slow, different biocompatible diluents and/or low molecular weight surfactants are used to reduce viscosity (e.g., 400 Mw PEG). MPs are characterized by SEM for size after passage through 19, 21, 23, and 25G needles. Bioactivity of the RLX MPs and DEX MPs post-needle passage are confirmed as previously described.

Stability testing is performed on three separate batches of particles according to ICH Harmonized Tripartite Guidelines. Long-term stability is evaluated at 25±2° C./60%±5% relative humidity over 18 months and accelerated stability is evaluated at 40±2° C./65%±5% relative humidity over 12 months. MPs are analyzed by DLS for size as well as by SEM for size confirmation, shape and level of degradation. The in vitro activity of released RLX and DEX is determined over time as described above.

### Example 14. Pharmacokinetic/Biodistribution

RLX and DEX levels after IA injection of MPs are measured in local cartilage, fibrotic and synovial tissues, organs (lungs, liver, spleen, kidneys, heart, brain), knee (ACL, meniscus, cartilage), synovial fluid, and plasma in rats treated with single IA injection of 10.0 g/kg RLX, 10.0 μg/kg RLX MPs, 50 μg/kg DEX, 50 μg/kg DEX MPs, or saline. The animals are euthanized at 0.25, 0.5, 1, 4, and 12 hrs, as well as 1, 2, 7, 14, and 30 d after treatment. RLX concentrations are measured via ELISA and DEX by LC MS/MS (N=6 per time point; 3F+3M).

### Example 15. Pharmacokinetic/Biodistribution

The experimental groups and outcomes for the two efficacy studies are summarized in the following tables. Shoulder torque values at full ROM is initially assessed to establish a healthy baseline and then animals are normalized to these individual torque values during measurement of contracted ROM. The angle corresponding to a given torque value is presented as joint ROM. Shoulder contracture is established via internal humeral fixation as previously described and subsequent contracted shoulder ROM is measured. Immediately after joint remobilization, animals receive treatment via IA injection. A subset of animals (N=4) are euthanized at 1, 2, and 4 weeks post-injection, with all remaining animals euthanized at 8 weeks (N=8 for longitudinal ROM, N=4 per time point, 20 animals per group).

Table 1 details determining the ability of DEX MPs to potentiate drug-receptor modulation via a dose reduction study where the DEX MP dose is maintained constant and the RLX MP dose is reduced from 10 to 0.3 μg/kg. From this study, we will establish the minimum effective dose (MED) of RLX MPs when potentiated by DEX MPs, versus appropriate controls in Table 2.

TABLE 1
Synergistic dose-range finding study design with 8 Sprague Dawley rats (4F + 4M) for longitudinal
ROM measurement for 8 weeks and then euthanized for terminal analyses. An additional 4 animals
(2F + 2M) will be euthanized at 1, 2, and 4 weeks for terminal analyses. 20 animals total per group.

| Group | | | |
|---|---|---|---|
| (n = 20) | Potentiator | Antifibrotic | Expected Outcome |
| 1 | DEX-MPs^{†} | Vehicle | Dexamethasone control. Minimal ROM recovery as measured |
| | (50.0 | MPs^{‡} | by kinematics and significant contracture as measured by |
| | μg/kg) | | histology. Results similar to untreated negative controls in the |
| | | | preliminary data. Reduction in local inflammation |
| 2 | DEX MPs^{†} | RLX MPs^{†} | Return to full ROM, no significant shoulder contracture, |
| | (50.0 | (10.0 | reduction of fibrosis histologically. Architecture similar to healthy |
| | μg/kg) | μg/kg) | contralateral shoulder control. More rapid return to full ROM and |
| | | | reduction of contracture than with RLX MPs alone (preliminary |
| | | | data). |
| 3 | DEX MPs^{†} | RLX MPs^{†} | Return to full ROM, unlike ineffective finding with 3.0 μg/kg in |
| | (50.0 | (3.0 | the preliminary data, no significant shoulder contracture, |
| | μg/kg) | μg/kg) | reduction of fibrosis histologically. Architecture similar to healthy |
| | | | contralateral shoulder control. More rapid return to full ROM and |
| | | | reduction of contracture than with RLX MPs alone (preliminary |
| | | | data). |
| 4 | DEX MPs^{†} | RLX MPs^{†} | Partial return to full ROM, decreased shoulder contracture, |
| | (50.0 | (1.0 | limited reduction of fibrosis histologically. Reduction in local |
| | μg/kg) | μg/kg) | inflammation. |
| 5 | DEX MPs^{†} | RLX MPs^{†} | Partial return to full ROM, decreased shoulder contracture, |
| | (50.0 | (0.3 | limited reduction of fibrosis histologically. Reduction in local |
| | μg/kg) | μg/kg) | inflammation. |

^{†}Values maybe adjusted by results obtained in Aims 1 and 2.
^{‡}equivalent polymer mass as highest respective dose.

TABLE 2
Study design 2 with 8 Sprague Dawley rats (4F + 4M) for longitudinal ROM measurement
for 8 weeks and then euthanized for terminal analyses. An additional 4 animals (2F +
2M) will be euthanized at 1, 2, and 4 weeks for terminal analyses. 20 animals total per
group. A single RLX injection control is not included as it was previously shown inefficacious.

| | | Dose & | |
|---|---|---|---|
| Group | | Regimen | |
| (n = 20) | Treatment | (50 μL) | Expected Outcome |
| 1 | Saline | 5 injections | Negative control. Minimal ROM recovery as measured by |
| | | | kinematics and significant shoulder contracture as measured by |
| | | | histology. Results similar to untreated negative controls in |
| | | | preliminary data. |
| 2 | RLX | 2.0 μg/kg^{†}; | Positive control. Injections every other day for 10 days. Return |
| | | 5 injections | to full ROM, no significant shoulder contracture. Increased |
| | | | inflammation at the joint due to repeated injections. |
| 3 | DEX | 10 μg/kg^{†}; | Clinical comparison control. Limited/negligible recovery of |
| | | 5 injections | ROM as measured by kinematics, with significant shoulder |
| | | | contracture as measured by histology. Results similar to Group 1. |
| 4 | Vehicle | Single | Microparticle control. Minimal ROM recovery as measured by |
| | MP | injection^{‡} | kinematics and significant contracture as measured by histology. |
| | | | Results similar to Group 1. Assessment of localized |
| | | | polymer/microparticle toxicity. |
| 5 | RLX MP | ^{§}MED_{RLX} | Limited/negligible recovery of ROM as measured by kinematics, |
| | | | with significant shoulder contracture as measured by histology. |
| | | | Results similar to Group 1 or sIA RLX in previous publication |
| 6 | DEX MP | 50 μg/kg^{†} | Minimal ROM recovery as measured by kinematics and |
| | | | significant contracture as measured by histology. Results |
| | | | similar to untreated negative controls in the preliminary |
| | | | data section. Reduction in local inflammation. |
| 7 | RLX MP | ^{§}MED_{RLX} | Return to full ROM, no significant shoulder contracture, |
| | DEX MP | 50 μg/kg^{†} | reduction of fibrosis histologically. Results similar to healthy |
| | | DEX | contralateral shoulder control. Return to full ROM and |
| | | | reduction of contracture will occur faster than with RLX |
| | | | MPs alone. Inflammation in shoulder will be reduced. |

Route of administration (Rte).
^{†}Values maybe adjusted by results obtained in Aims 1 and 2.
^{‡}Equivalent polymer mass at highest respective dose.
^{§}Values based on results from study detailed in Table 1.

Following contracture and joint re-mobilization, twice-weekly assessment of external and internal shoulder ROM are performed using the baseline torque levels initially measured. ROM measurements of the contralateral shoulder serves as internal, healthy controls to reduce total animals used. Voluntary wheel running behavior is used as a correlate of physical activity for each animal. Analyses will be performed at baseline, after animal habituation and prior to arthrofibrosis induction, and at 1, 2, 4, and 8 weeks after joint remobilization. The number of running cycles, total distance traveled, peak speed, and average duration from the start of running to the peak speed will be assessed.

The extent of arthrofibrosis present in the shoulder joint is assessed upon euthanasia of a subset of each group at 1, 2, 4 and 8 weeks post-injection. Synovial fluid is collected to determine the concentrations of RLX (R&D Systems), MMP-1/3/9/13 (R&D Systems), TIMP-1 (R&D Systems), Cartilage Oligomeric Matrix Protein (COMP) (LifeSpan BioSciences), and tartrate-resistant acid phosphatase (TRAP)-5b (ImmunoDiagnostic Systems Inc.) using ELISAs. Cytokine levels are also assessed in the synovial fluid and serum to identify any markers of potential inflammation or toxicity (R&D Systems). Increased levels of COMP, TRAP-5b, and MMPs are markers of fibrosis reduction. Both shoulders (treated and contralateral) are harvested and fixed, decalcified, paraffin-embedded, and sectioned for histological analysis (e.g. H&E, Safranin-O, Masson's Trichrome). Group 4 (Table 1) shoulder histology is analyzed for signs of toxicity and inflammation associated with polymer alone. Immunohistochemical staining of the synovial lining, fibrotic tissue, and cartilage tissue sections is used for quantification of: 1) RLX71; 2) collagen types I, II and III using a peroxidase-anti-peroxidase method; 70,72 3) MMP-1, 3, 9, and 1316; 4) as described by Zhang et al.; 5) TRAP-5b as described by Furuya; and 6) the number and distribution of myofibroblasts. The staining intensity for collagen I/II/III, MMPs, aggrecan, TRAP-5b, and myofibroblasts is evaluated via blinded image quantification. Finally, the mRNA from tissue surrounding the shoulder joint is collected through mechanical homogenization and subsequent total mRNA extraction. Gene expression of RXFP1 as well as regulation-associated proteins are assessed to determine the impact of DEX potentiation on RLX activity in arthrofibrotic tissue. RNA-sequencing is performed to analyze changes in gene transcripts of synovial tissue and peri-articular tissue with and without RLX, DEX, and RLX+DEX treatment. Expression profiles of treated rats and vehicle-treated rats is compared longitudinally to identify key transcriptomic players in the antifibrotic and RLX-potentiation cascade.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patent applications, patents, publications and other references mentioned in the specification are indicative of the levels of those of ordinary skill in the art to which the invention pertains and are each incorporated herein by reference. The references cited herein are not admitted to be prior art to the claimed invention.

Unless otherwise defined, all technical and scientific terms used herein have the same meaning as commonly understood by one of ordinary skill in the art to which this invention belongs. In the case of conflict, the present specification, including definitions, will control.

The use of the articles “a”, “an”, and “the” in both the description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about”, “approximately”, or “approximate”, when used in connection with a numerical value, means that a collection or range of values is included. For example, “about X” includes a range of values that are ±20%, ±10%, ±5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method, comprising:
co-administering to a subject in need thereof a small molecule that increases expression, concentration, or cell surface density of a therapeutic target, and a ligand of the therapeutic target, wherein the therapeutic effect of the ligand is potentiated by administration of the small molecule.

2. A method according to claim 1, wherein the therapeutic target is a relaxin receptor.

3. A method according to claim 1, wherein the small molecule potentiator increases the cell surface expression of RXFP1.

4. A method according to one of claims 1-3, wherein the ligand is relaxin or a fragment thereof that binds to and induces relaxin/RXFP1 signaling.

5. A method according to one of claims 1-3, wherein the ligand is a small molecule RXFP1 agonist.

6. A method according to claim 5, wherein the ligand is 2-[[2-(1-methylethoxy)benzoyl]amino]-N-[3-[(trifluoromethyl) sulfonyl]phenyl]-benzamide or an analog thereof.

7. A method according to one of claims 1-6, wherein the small molecule potentiator is a corticosteroid.

8. A method according to claim 7, wherein the potentiator is selected from the group consisting of dexamethasone, methylprednisolone, cortisone, hydrocortisone, betamethasone, prednisolone, prednisone, triamcinolone, and fludrocortisone.

9. A method according to one of claims 1-8, wherein the small molecule and the ligand are administered simultaneously.

10. A method according to one of claims 1-8, wherein the small molecule is administered prior to administration of the ligand.

11. A method according to one of claims 1-8, wherein the small molecule is administered following administration of the ligand.

12. A method according to one of claims 1-11, wherein the small molecule and ligand are delivered by the same route of administration;

13. A method according to one of claims 1-11, wherein the potentiator and ligand are delivered by different routes of administration.

14. A method according to one of claims 1-19, wherein the small molecule and the ligand are contained within the same carrier.

15. A composition comprising a small molecule that increases expression, concentration, or cell surface density of a therapeutic target, and a ligand of the therapeutic target, wherein the therapeutic effect of the ligand is potentiated by administration of the small molecule

16. A composition according to claim 15, wherein the small molecule potentiator and ligand are contained within the same carrier.

17. A composition according to claim 16, wherein the carrier is selected from the group consisting of a microparticle, a nanoparticle, a mesh, a polymeric buttress, a hydrogel, a lotion, a cream, a viscosupplement, and a solution;

18. A composition according to claim 16, wherein the carrier is a microparticle comprising an aliphatic polyester, ligand, and small molecule potentiator, wherein (i) said microparticles have a diameter of 1-100 μm; (ii) said ligand is present in an amount that is 0.01-25% of the total mass; (iii) said small molecule is present in an amount that is 0.01-25% of the total mass; (iv) said aliphatic polyester has a molecular weight of between 10,000 and 200,000 Daltons.

19. A composition according to one of claims 15-18, wherein the therapeutic target is a relaxin receptor.

20. A composition according to claim 19, wherein the small molecule potentiator increases the cell surface expression of RXFP1.

21. A composition according to one of claims 15-18, wherein the ligand is relaxin or a fragment thereof that binds to and induces relaxin/RXFP1 signaling.

22. A composition according to one of claims 15-18, wherein the ligand is a small molecule RXFP1 agonist.

23. A composition according to claim 22, wherein the ligand is 2-[2-(1-methylethoxy)benzoyl]amino]-N-[3-[(trifluoromethyl) sulfonyl]phenyl]-benzamide or an analog thereof.

24. A composition according to one of claims 15-23, wherein the small molecule potentiator is a corticosteroid.

25. A composition according to claim 24, wherein the potentiator is selected from the group consisting of dexamethasone, methylprednisolone, cortisone, hydrocortisone, betamethasone, prednisolone, prednisone, triamcinolone, and fludrocortisone.
